(19) United States
(12) Patent Application Publication
(10) Pub. No.: US 2005/0250215 A1
(43) Pub. Date: Nov. 10, 2005

(54) MEASUREMENT OF MELANOCORTIN PEPTIDES AND USES THEREOF

(76) Inventors: Kathleen Grace Mountjoy, Auckland (NZ); Jenny Wu Chia-Shan, Auckland (NZ)

Correspondence Address: FISH & NEAVE IP GROUP;ROPES & GRAY LLP, ONE INTERNATIONAL PLACE, BOSTON, MA, 02110-2624, US

(21) Appl. No.: 10/517,684

(22) PCT Filed: Jun. 11, 2003
(86) PCT No.: PCT/IB03/02641

§ 371 Date: Jun. 6, 2005

(30) Foreign Application Priority Data
Jun. 11, 2002 (NZ) 519504
Aug. 23, 2002 (AU) 2002951020

(51) Int. Cl. G06F 19/00; G01N 33/48; G01N 33/50; G01N 33/00

(52) U.S. Cl. 436/86; 702/19

(57) ABSTRACT

The present invention relates to melanocortin peptides and to methods that utilize melanocortin peptides, their measurement, their receptors and biological response systems for the risk assessment and diagnosis of disease. The biological response systems are also utilized to screen for compounds that act as agonists or antagonists of melanocortin receptors.

### TECHNICAL FIELD

The present invention relates to melanocortin peptides and to methods that utilise melanocortin peptides, their measurement, their receptors and biological response systems for the risk assessment and diagnosis of disease. The biological response systems are also utilised to screen for compounds that act as agonists or antagonists of melanocortin receptors.

### BACKGROUND

Obesity and type 2 diabetes are major health problems worldwide and are a major threat to health and well-being. Over the last few years significant advances have been made with respect to the molecular determinants of energy balance and insulin resistance. Critical elements of this control system are hormones secreted in proportion to body fat, including leptin and insulin, and their central nervous system targets such as neuropeptide Y and the hypothalamic melanocortin system. Recently proopiomelanocortin and MC4-R have been identified as important targets mediating leptin's activities in the hypothalamus.

Pro-opiomelanocortin (POMC), produced in the pituitary and brain and to a lesser extent in numerous peripheral tissues including skin, pancreas and testis, is the large precursor protein from which melanocortin peptides α-melanocyte stimulating hormone (MSH) and adrenocorticotropin (ACTH) and fragments thereof, are derived. The products of POMC undergo a series of complex, tissue specific, processing events such as further proteolytic cleavages, phosphorylation, α-amidation and NH₂-terminal acetylation which influence their biological activities. ACTH₁₋₁₃NH₂ exists as α-MSH and desacetyl-α-MSH, α-MSH, which is acetylated at the N-terminus and amidated at the COOH terminus, is a post translationally modified derivative of ACTH₁₋₁₃NH₂ (desacetyl-α-MSH). The acetylation reaction to form α-MSH is associated with the secretory process: Its highest activity is present in the pituitary gland and certain brain regions.

The functional significance of N-terminal acetylation of ACTH₁₋₁₃ in the central nervous system is unknown. N-terminal acetylation of desacetyl-α-MSH to form α-MSH enhances some activities of ACTH₁₋₁₃ and virtually ellminates others. α-MSH injected daily to rats is 10-100 fold more effective than desacetyl-α-MSH at increasing pigmentation, arousal, memory, attention, and excessive grooming. Desacetyl-α-MSH, however, is more effective than α-MSH at blocking opiate analgesia and opiate receptor binding in vivo. α-MSH and desacetyl-α-MSH also differentially affect feeding and weight gain. Weight gain of agouti obese mice is increased by subcutaneously administered desacetyl-α-MSH, as is food intake and fat pad weight, but α-MSH injections do not significantly increase food intake or body weight.

Despite advances in the understanding of energy homeostasis, efforts have not yielded clinically applicable parameters with which to predict or diagnose pathological imbalances that lead to obesity. There is a need therefore for methods which would assist in the analysis and monitoring of energy metabolism, feeding and weight gain patterns and diagnosis and/or prognosis of associated disorders and diseases.

It is an object of the present invention to ameliorate at least some of the disadvantages of the prior art methods, or at least provide useful alternatives.

### SUMMARY OF THE INVENTION

According to a first aspect there is provided a method for assessing feeding and/or weight gain pattern in a subject comprising the measurement of a melanocortin peptide in a sample obtained from said subject and comparison of the measured value with a reference value.

According to a second aspect there is provided a method for predicting risk of obesity in a subject comprising the measurement of a melanocortin peptide in a sample obtained from said subject and comparison of the measured value with a reference value

According to a third aspect there is provided a method for diagnosing imbalance in energy homeostasis in a subject comprising the measurement of a melanocortin peptide in a sample obtained from said subject and comparison of the measured value with a reference value.

According to a fourth aspect there is provided a method for diagnosing obesity in a subject comprising the measurement of a melanocortin peptide in a sample obtained from said subject and comparison of the measured value with a reference value

According to a fifth aspect there is provided a method for screening medicaments for the adverse reactions of imbalance in energy homeostasis, feeding/weight gain patterns or obesity in a subject to whom the medicament has been administered comprising the measurement of a melanocortin peptide in a sample obtained from said subject, and comparison of the measured value with a reference value.

According to a sixth aspect there is provided a method for screening foods and/or diets for the adverse reactions of imbalance in energy homeostasis, feeding/weight gain patterns or obesity in a subject to whom the medicament has been administered comprising the measurement of a melanocortin peptide in a sample obtained from said subject, and comparison of the measured value with a reference value. Preferably, the melanocortin peptide measured is either α-MSH or desacetyl-α-MSH.

Preferably the melanocortin peptide measured is α-MSH or desacetyl-α-MSH.

According to a seventh aspect there is provided a method for assessing feeding and/or weight gain pattern in a subject comprising the measurement of at least two melanocortin peptides in a sample obtained from said subject, the calculation of the ratio of the measured melanocortin peptides and comparison of the value of the ratio with a reference value.

According to an eighth aspect there is provided a method for predicting risk of obesity in a subject comprising the measurement of at least two melanocortin peptides in a sample obtained from said subject, the calculation of the ratio of the measured melanocortin peptides and comparison of the value of the ratio with a reference value.

According to a ninth aspect there is provided a method for diagnosing obesity in a subject comprising the measurement of at least two melanocortin peptides in a sample obtained from said subject, the calculation of the ratio of the measured melanocortin peptides and comparison of the value of the ratio with a reference value.

According to a tenth aspect there is provided a method for diagnosing imbalance in energy homeostasis in a subject comprising the measurement of at least two melanocortin peptides in a sample obtained from said subject, the calculation of the ratio of the measured melanocortin peptides and comparison of the value of the ratio with a reference value.

According to an eleventh aspect there is provided a method for screening medicaments for the adverse reactions of imbalance in energy homeostasis, feeding/weight gain patterns or obesity in a subject to whom the medicament has been administered comprising the measurement of at least 2 melanocortin peptides in a sample obtained from said subject, the calculation of the ratio of the measured melanocortin peptides, and comparison of the value of the ratio with a reference value.

According to a twelfth aspect there is provided a method for screening foods and/or diets for the adverse reactions of imbalance in energy homeostasis, feeding/weight gain patterns or obesity in a subject to whom the medicament has been administered comprising the measurement of at least 2 melanocortin peptides in a sample obtained from said subject, the calculation of the ratio of the measured melanocortin peptides, and comparison of the value of the ratio with a reference value.

Preferably the melanocortin peptide ratio calculated is the ratio of desacetyl-α-MSH to α-MSH.

It will be understood that the melanocortin peptides can also be measured by a biological response system in which the resulting profile of response parameters is predictive of the risk of developing obesity or diagnostic of obesity, imbalance in energy homeostasis or disturbance in feeding/weight gain patterns.

According to a thirteenth aspect there is provided a method of assessing risk of developing obesity, diagnosing obesity or diagnosing an imbalance in energy homeostasis or disturbance in feeding/weight gain patterns in a subject, comprising:
a. measuring the amount of α-MSH and desacetyl-α-MSH in a sample obtained from the subject, either directly or by subtraction of one of the amount of α-MSH or desacetyl-α-MSH from a measured amount of total MSH in the sample,
b. calculating the ratio of the amounts of desacetyl-α-MSH to α-MSH.
c. comparing the ratio of desacetyl-α-MSH to α-MSH with a reference ratio.

The methods of the present invention may utilise quantitative measurements of melanocortin peptides and may do so on intact samples of after separation of melanocortin peptides, in particular desacetyl-α-MSH and α-MSH. Preferably, the separation procedure is selected from chromatography, electrophoresis, immunocapture, affinity capture including receptor-ligand capture or other affinity capture, and the like. It is also preferable that the quantitation procedure is selected from immunoassay including RIA, ELISA, Western blot, immunoprecipitation, and affinity capture, including receptor-ligand capture, peptide-nucleotide affinity capture or other affinity capture, and catalytic reaction-based assay, and the like. More preferably, the separation of the melanocortin peptide is by chromatography and the quantitation is performed by an immunoassay. The chromatographic method described herein, only as an example of such a procedure, is HPLC and the exemplary immunoassay described is RIA. All these detection, quantitation ans separation techniques are described in detail in standard laboratory manuals which will be known to those skilled in the art.

According to a fourteenth aspect there is provided a method of monitoring treatment for obesity or for imbalance in energy homeostasis and/or disturbance in feeding/weight gain pattern in a subject comprising contacting a sample obtained from the subject having such treatment with a biological response system wherein the resulting profile of response parameters is indicative of the effect of such treatment on obesity or imbalance in energy homeostasis and/or disturbance in feeding/weight gain pattern.

According to a fifteenth aspect there is provided a method of assessing the risk of developing obesity or developing and/or having an imbalance in energy homeostasis and/or disturbance in feeding/weight gain pattern in a subject comprising analysing the profile of response parameters in a sample from a test subject by comparing it with
(i) the profile of a sample from a normal subject and
(ii) the profile of a sample from an obese subject or a subject with an imbalance in energy homeostasis and/or disturbance in feeding/weight gain pattern,
   wherein resemblance of the profile of the sample obtained from the test subject to that of the profile in (ii) above, is indicative of that subject being at risk of developing obesity or developing and/or having an imbalance in energy homeostasis and/or disturbance in feeding/weight gain pattern.

Preferably the subject is a mammal and even more preferred is a human subject. Levels of melanocortin receptors (eg. α-MSH and/or desacetyl-α-MSH) may vary with age and between gender. Therefore it is appropriate to compare quantitative levels, ratios and/or biological response parameters in test subjects with those for appropriately sex and age matched control subjects. Of course internal control values may also be used, particularly if monitoring effects of certain drugs or foods, or if monitoring effects of treatments as described herein.

According to a sixteenth aspect there is provided a method of determining the melanocortin peptide status of a sample comprising contacting the sample with a biological response system wherein the resulting profile of response parameters produced by the biological response system indicates the melanocortin peptide status of the sample.

Preferably the sample is a biological fluid such as for example whole blood, plasma, serum, saliva, sweat, urine, amniotic fluid, cord blood, cerebrospinal fluid and the like. The sample may also consist of tissue culture fluid or other medium in case where use is made of cells or tissues in vitro as biological response systems.

According to a seventeenth aspect there is provided a method of screening for a compound which acts as agonist or antagonist of a melanocortin receptor comprising treating a biological response system with a test compound and measuring the resulting profile of response parameters that are indicative of agonist or antagonist activity to the melanocortin receptor.

According to a eighteenth aspect there is provided a method of screening for a compound that is useful in the treatment of obesity comprising exposing a biological response system to a test compound and measuring the resulting profile of response parameters that are indicative of the desired response for the treatment of obesity.

According to a ninteenth aspect there is provided a method of screening for a compound that is useful in the treatment of an imbalance in energy homeostasis or a disturbance in feeding/weight gain patterns comprising exposing a biological response system to a test compound and measuring the resulting profile of response parameters that are indicative of the desired response for the treatment of an imbalance in energy homeostasis or a disturbance in feeding/weight gain patterns.

Preferably, the biological response system is an in vitro cell or organ sample or culture capable of responding to melanocortin peptides. The preferred in vitro cells are cultures of primary rat osteoblasts, or the UMR106.06 rat osteosarcoma cell line, or the GT1-7 mouse hypothalamic cell line. Any cell line or primary culture of cells that expresses melanocortin receptors, or any combination of such cell lines, may also be used as an in vitro biological response system. Some of these cell lines are 3T3-L1 adipocytes, melanocytes, L6 myocytes, B16 melanoma cells, and anterior pituitary cell cultures. Any cell line or primary culture of cells that express melanocortin receptors, or any combination of such cell lines, that are capable of producing a differential response that distinguishes obese individuals, or individuals at risk of developing obesity, or individuals suffering from an imbalance in energy homeostasis or disturbance in feeding/weight gain patterns, from normal individuals may be used as an in vitro biological response system. As the given list is not exhaustive of cell lines or primary cell cultures that express melanocortin receptors, the in vitro biological response system described herein is not limited to the use of these. The biological response system may also be an in vivo system. Examples of in vivo systems include the hypothalamus of a mammal and/or other tissue(s) that are capable of responding to melanocortin peptides.

Of course, it will be understood that a whole animal may be used as an in vivo biological response system. In the case where a whole animal is used as an in vivo biological response system the response parameters may be feeding frequency and/or body weight gain. Further, samples may be introduced in to the animal biological response system, and tissues and/or organ samples may be obtained from the animal biological response system, which samples may be analysed for the relevant response parameters.

The preferred response profile or fingerprint is one or more proteins or cellular events which differentiate between normal individuals and those at risk of developing obesity, or those suffering from obesity, or those with an imbalance in energy homeostasis, or disturbance in feeding/weight gain patterns.

The preferred response parameters are proteins expressed by the biological response system. Proteins expressed by the biological response system includes but are not limited to stress proteins such as heat shock protein homologue, enzymes such as glyceraldehyde-3-phosphate-dehydrogenase, aldo-keto reductase, citrate synthase, creatine kinase, pyruvate synthase alpha-chain, f1 ATPase beta-chain, and cytoskeletal proteins such as tubulin beta-chain. Other proteins which may be used as response parameters include but are not limited to proteins involved in the melanocortin peptidergic axis, proteins involved in signalling pathways, enzymes, and membrane-bound proteins. Extracellular effector molecules may also be suitable response parameters.

### BRIEF DESCRIPTION OF THE FIGURES

FIG. 1. Displacement of ¹²⁵/-α-MSH bound to rabbit antiserum (1:9000) by increasing amounts of melanocortin peptides. Insert: HPLC separation of α-MSH and desacetyl-α-MSH peptides.

FIG. 2. Alpha-MSH but not desacetyl-α-MSH administered i.c.v. significantly decreased food intake. Food intake was measured over 3 h following lateral ventricle injections of vehicle (PBS), 10 μg□ α-MSH, or 10 μg desacetyl-α-MSH to food deprived Wistar rats. (PBS, n=9; α-MSH, n=7; desacetyl-α-MSH, n=10). □Alpha-MSH significantly decreased food intake to 70% of PBS treated control (*, significantly different from PBS, p<0.05, one way ANOVA). Desacetyl-α-MSH has no significant effect on feeding, but there was a trend for a reduction in food intake.

FIG. 3. A higher dose of desacetyl-α-MSH compared to α-MSH administered i.c.v. significantly decreased food intake.

Food intake was measured over 3 h following lateral ventricle injections of vehicle (PBS), 10 μg α-MSH, or 50 μg desacetyl-α-MSH to food deprived Wistar rats. (PBS, n=11; α-MSH, n=11; desacetyl-α-MSH, n=11). (*, significantly different from PBS<p<0.05, one way ANOVA).

FIG. 4. Desacetyl-α-MSH significantly slowed body weight change in neonatal rats. Neonatal rats were injected subcutaneously with PBS (n=36), α-MSH (n=27) or desacetyl-α-MSH (n=27) (0.3 μg/g body weight/day) for their first 14 days of life. There were no significant differences in body weight over 14 days between PBS and α-MSH treated pups. Neonatal rats treated with desacetyl-α-MSH for 14 days grew significantly slower than either PBS or α-MSH treated pups (p<0.05, GLM repeated measures analysis of variance, SAS system).

FIG. 5 RT-PCR shows MC2-R, MC4-R and MC5-R expression in primary rat osteoblast cells. Lane 2, MC2-R PCR product (290 p); lane 4, MC4-R PCR product (554 bp); lane 6, MC5-R PCR product (290 bp); controls of specificity were the absence of RT in the reverse transcription reaction mixture (lane 3, MC2-R; lane 5, MC4-R; lane 7, MC5-R). The primers used are shown in Table 1. The PCR products were run on a 2% agarose gel alongside a HindII-EcoRI digested lambda DNA molecular weight marker (lane 1).

FIG. 6 Northern blot analysis showed MC4-R mRNA transcripts in primary rat osteoblasts.

Poly (A⁺) mRNA (5 μg) from rat brain (lane 1) and primary rat osteoblasts (lane 2) were separated by formaldehyde-agarose gel electrophoresis (1.2%), transferred to a nylon membrane and probed with a ³²P labeled specific rat MC4-R DNA fragment. A digital image was obtained with a Storm imaging system screen and scanner. An RNA ladder was run on the gel and used to determine the mRNA sizes (2.0-2.6).

FIG. 7 Ribonuclease Protection Assay shows MC4-R mRNA expression in UMR106.06 and primary rat osteoblast cells. Lane 2, full length rat MC4-R riboprobe (562 bp), probe incubated with: lane 3, 1 μg/ml RNase A and 50 U Rnase T1; lane 4, 10 μg tRNA; lane 5, 10 μg rat brain poly (A⁺) mRNA, lane 6, 10 μg primary rat osteoblast poly (A⁺) mRNA; lane 7, 10 μg UMR106.06 poly (A⁺) mRNA. The labeled fragments were run on a 6% polyacrylamide gel alongside a radiolabeled 123 bp DNA Ladder (GIBCO BRL) (lane 1). The data shown are representative of three independent experiments.

FIG. 8 Alpha-MSH stimulation of rat primary osteoblast proliferation. Growth arrested primary rat osteoblasts were stimulated with increasing doses of α-MSH and [³H] thymidine uptake (a) and changes in cell number (b) measured. Data are expressed as mean±SEM. Significant difference from control; *=p<0.04, **p<0.001

FIG. 9. Desacetyl-α-MSH and ACTH1-24 antagonise α-MSH stimulated stimulation of thymidine incorporation into culltures of rat primary osteoblasts. Growth arrested primary rat osteoblasts were stimulated with either 10⁻⁷M or 10⁻⁸M α-MSH alone (a,b), 10⁻⁷M desacetyl-α-MSH alone (a), ACTH₁₋₂₄ alone (b), or combinations of α-MSH and desacetyl-α-MSH (a) or α-MSH and ACTH₁₋₂₄ (b) and [³H] thymidine uptake measured. Data are expressed as mean±SEM. Significant difference from control; *=p<0.04, **p<0.001

FIG. 10. Biphasic Dose response curve for treatment of UMR106.06 with alpha-MSH. UMR106,06 rat osteosarcoma cells were stimulated with 10⁻⁶ to 10⁻¹² alpha-MSH and the [³H] thymidine uptake measured.

FIG. 11. Dose response curve for treatment of UMR106.06 with desacetyl-alpha-MSH. UMR106.06 rat osteosarcoma cells were stimulated with 10⁻⁶ to 10⁻¹² desacetyl-alpha-MSH and the [³H] thymidine uptake measured.

FIG. 12 Effects of alpha-MSH on Thymidine incorporation in Chondrocyte monolayers. The figure shows increased thymidine incorporation (interpreted as increased cell proliferation) in response to stimulation by alpha-MSH.

### DESCRIPTION OF THE PREFERRED EMBODIMENT

The present invention is based on a surprising observation that the balance/abundance/status of MSH peptides in the circulation, may correlate with, and be predictive of, the development of an imbalance in energy homeostasis, disturbance in feeding/weight gain patterns and ultimately obesity.

Just as the measurement of “good” (HDL) and “bad” (LDL) cholesterol predicts cardiovascular risk, we have discovered that the balance, le. the ratio, of melanocortin peptides α-MSH and desacetyl-α-MSH is particularly predictive and/or diagnostic of imbalances in energy homeostasis, disturbances in feeding/weight gain patterns and ultimately obesity. However, absolute level of individual, or combination of, MSH peptides will also serve this purpose.

A novel approach described herein involves the use of a biological response system that processes stimulus through melanocortin receptors, and which outputs information through various response parameters. Of course, simple quantitative measurement of MSH peptides in samples of biological fluids, such as antibody-based methods, and the use of such data to determine ratios of MSH peptides, may also be used in the prognostic/diagnostic methods of the present invention. The biological response system may be used in conjunction with the simple quantitative measurements, to enhance the power of the methods described herein.

The measurement of specific MSH peptides in subject's plasma or other biological fluids, as described herein in one embodiment, follows extraction and fractionation using high pressure liquid chromatography (HPLC), followed by classical RIA, according to modified methods described in the literature (Facchinetti, F., Bernasconi, S., lughetti, L., Genazzani, A. D., Ghizzoni, L., Genazzani, A. R. Changes in dopaminergic control of circulating melanocyte-stimulating hormone-related peptides at puberty. Pediatric Research 38; 91-94, 1995; Mauri, A., Volpe, A., Martellotta, M. C., Barra, V., Plu, U., Angioni, G., Angioni, S., Argiolas, A. α-Melanocyte-stimulating hormone during human perinatal life. J Clln Endocrinol Metab 77: 113-117, 1993: Mauri, A., Martellotta, M. C., Melis, M. R., Caminiti, F., Serri, F., Fratta, W. Plasma alpha-melanocyte-stimulating hormone during the menstrual cycle in women. Hormone Research 34: 66-70, 1990). This approach was adopted initially to verify the identity of the MSH peptides and ascertain the functionality of the immuno-based and biological response methodology. Simple quantitative immuno-assay type methods for measuring MSH peptides in a sample can be employed with equivalent results.

Analysis of the abundance of and, particularly the ratios of, α-MSH and desacetyl-α-MSH in blood circulation or other body fluid containing MSH peptides, are novel developments in the field of prediction and/or diagnosis of predisposition to obesity.

For the purposes of the invention herein described, the term “biological response system” includes any whole animal, organ, tissue or cell which is able to respond to a melanocortin peptide or an effector molecule generated by a response to a melanocortin peptide.

For the purposes of the invention herein described, the term “response parameter” includes a cellular product (which may be a protein, nucleic acid, lipid, carbohydrate or a combination of these), or a measurable cellular event, resulting from interaction of the biological response system with a melanocortin peptide, for example cell proliferation, cell cycle progression, cell differentiation and the like, mass spectrometry or currently commercially available gene expression arrays may be used to monitor these response parameters, among other techniques.

Not wishing to be bound by any particular theory, when the biological response system is treated with melanocortin peptides, or a sample containing melanocortin peptides, the profile or “fingerprint” of response parameters resulting from melanocortin receptor stimulus also reflects the melanocortin peptide balance/abundance/status of the sample. A comparison of the fingerprints of response parameters resulting from normal subjects and obese individuals, or individuals with an imbalance in energy homeostasis and/or disturbance in feeding/weight gain patterns provides additional information, by way of profile databases, that may be used to predict imbalance in energy homeostasis and/or disturbance in feeding/weight gain patterns or the risk of onset of obesity or that may be diagnostic of these conditions.

For the purpose of the invention described herein, the term “profile” or “fingerprint of response parameters” is a reference to one or a plurality of response parameters that may be ascertained by various techniques, which are indicative of an imbalance in energy homeostasis and/or disturbance in feeding/weight gain patterns, obesity or the risk of onset of obesity.

The response parameters that are profiled in the biological response systems may be the result of a primary response by the system to stimulus by melanocortin peptides, or they may be the result of a secondary response following the primary response to melanocortin peptides. The response profile may be utilised to monitor treatments used for obesity.

The profiles may also be used to monitor the onset of obesity [, the efficacy of treatment, relapse or progression of or imbalance in energy homeostasis and/or disturbance in feeding/weight gain patterns. The profile of parameters may therefore be adopted as a clinician's tool to assess risk of developing disease, diagnose disease, monitor disease and monitor treatment of disease.

The biological response system is also useful to screen for compounds that are effective in the treatment of imbalances in energy homeostasis and/or disturbances in feeding/weight gain patterns or obesity. The system would also be useful to screen for compounds that act as agonists or antagonists of melanocortin receptors. The response to test compounds, reflected in the resulting profile of response parameters, may be monitored by mass spectrometry or currently commercially available gene expression arrays, among other techniques. Such compounds are potential candidates for the treatment or prevention of obesity, or an imbalance in energy homeostasis, or a disturbance in feeding/weight gain patterns, or other metabolic imbalances brought about by disturbances in melanocortin peptide balance/abundance/status and the resultant receptor response.

Preferred embodiments of the invention will now be described by way of example only with reference to the following examples.

### EXAMPLES

### Example 1.

### Method for Separation and Detection/Quantitation of α-MSH and Desacetyl-α-MSH in Plasma Extracts

1.1. Extraction of Plasma Using Sep-Pak C18 Cartridge

Plasma (1-2 mL rodent or 10-20 mL human) was collect on ice and equal volume of 0.1M HCl add, and left for 30 minutes on ice. The plasma was spun for 30 minutes at 3300 rpm at 4° C. before use.

Sep Pak C18 cartridges (Waters Corporation, MA, USA) were pre-washed with 10 mL methanol followed by 10 mL phosphate buffered saline (PBS). Sample was loaded onto column at flow rate of 5-10 mL per minute. 3mL of 10% methanol in 0.5M acetic acid was run over to elute non-specific or interfering substances (5-10 mL per minute). MSH peptides were eluted with 9 mL 90% methanol in 0.5M acetic acid into silicanised tubes, then freeze dried to dryness with 900 μg polypep (Sigma-Aldrich, MO, USA) and 9 μL of 330 μM n-octyl-β-D-glucopyranoside (Sigma-Aldrich, MO, USA) added to each tube.

1.2 Separation ofα-MSH and Desacetyl-α-MSH Using HPLC

Freeze dried mixture (after Sep-Pak extraction) was reconstituted in 150 μL HPLC buffer (acetonitrile: 0.1% trifluoroacetic acid (TFA) mixed at a ratio of 18:82). The sample was spun in Eppendorf tube to remove any precipitated material before transferring the sample to HPLC.

100 μl of sample was injected onto HPLC C18 column (μ Bondpack, 39×300 mm, 10 μM size) and fractions collected by eluting with a linear gradient from 18-40% acetonitrile in 0.1% TFA at a flow rate of 1.5 mL/min. Fractions were collected into 6 mL siliconised glass kimble tubes each of which contained 15 μL of 10 mg/mL polypep and 1.5 μL of 330 μM n-octyl-β-D-glucopyranoside (Sigma-Aldrich, MO, USA). The fractions were freeze dried.

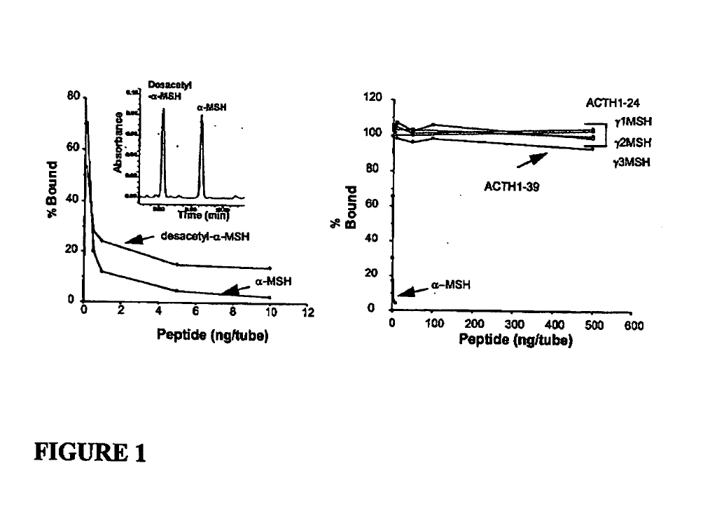

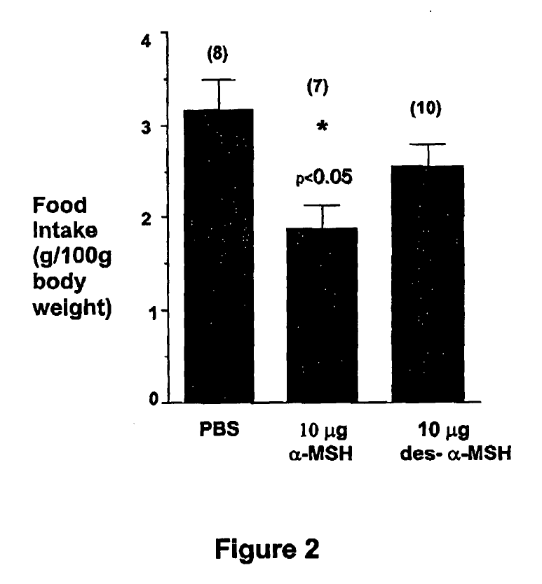

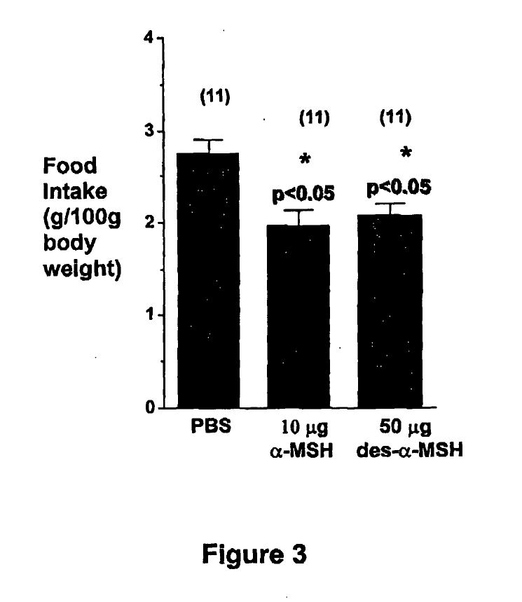

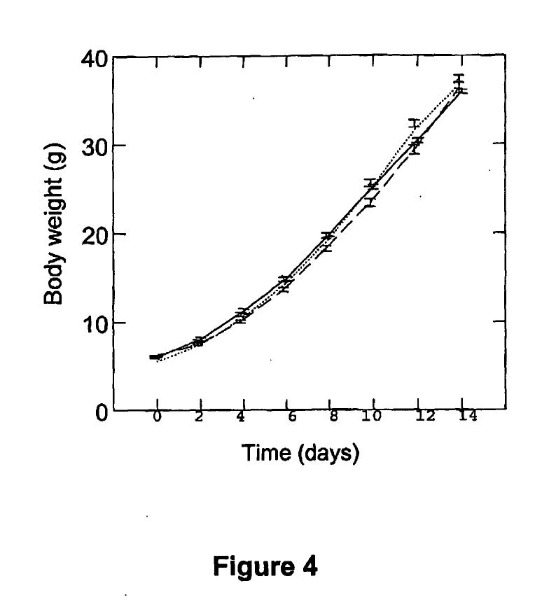

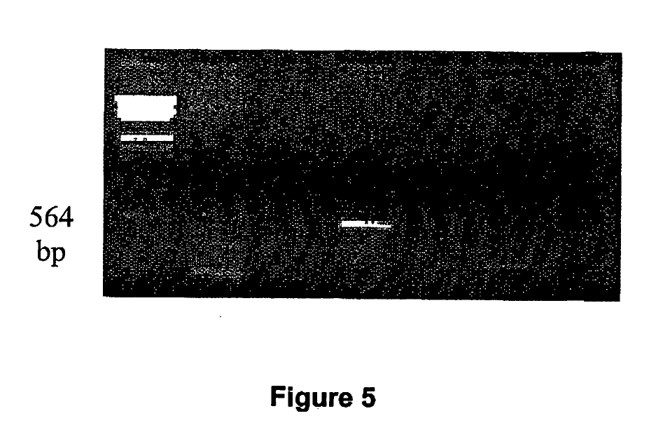

The retention times were: α-MSH, 8.6 minutes, and desacetyl-α-MSH, 6.5 minutes (FIG. 1: Insert). It will be appreciated by those skilled in the art that this separation technique is applicable to samples other than plasma extracts. In fact it will be applicable without significant alterations to any biological fluid containing MSH peptides as well as samples of purified MSH peptides.

The separated α-MSH and desacetyl-α-MSH peptides are then quantitated using a sensitive and specific immunoreactive assay.

1.3 Radioimmunoassay of MSH peptides.

α-MSH and desacetyl- α-MSH were obtained from Bachem AG, Hauptstrasse 144, Switzerland
   Alpha-MSH
Ac-Ser-Tyr-Ser-Met-Glu-His-Phe-Arg-Trp-Gly-Lys-Pro-Val-NH2 (Bachem # H-1075.0001)
   Desacetyl-Alpha-MSH
H-Ser-Tyr-Ser-Met-Glu-His-Phe-Arg-Trp-Gly-Lys-Pro-Val-NH2 (Bachem #H-4390.0001)

Freeze dried samples were reconstituted in RIA assay buffer (rodent—200 μL; human—300 μL). RIA assay buffer: 0.05 M phosphate buffer pH 7.4, 0.1 M NaCl, 0.5% BSA, 10 mM EDTA,

¹²⁵lα-MSH was diluted to 10,000 cpm in RIA assay buffer.

α-MSH standards were prepared in RIA assay buffer: 0.00075, 0.001, 0.0015, 0.002, 0.003, 0.004, 0.005, 0.0075, 0.01, 0.015 ng/100 μL.

Desacetyl-α-MSH standards were prepared in RIA assay buffer: 0.001, 0.002, 0.005, 0.01, 0.02, 0.05, 0.1, 0.5 ng/100 μL.

Assay procedure: tubes set up in duplicate with the following:

a) 100 μL standard or sample
b) 100 μL rabbit polyclonal antibody (KM4), 1:20,000 diluted in RIA assay buffer
c) Vortex and incubate overnight at 4° C.
d) Add 100 μL ¹²⁵l-α-MSH (10,000 cpm) to each tube
e) Vortex and incubate overnight at 4° C.
f) Prepare secondary antibody mix: 8% PEG 6000 in 0.01M PBS. 1% #2 sheep anti-rabbit gamma globulin, 0.025% normal rabbit serum.
g) Add 1 mL secondary antibody mix to each tube
h) Vortex and incubate 1 hour at room temperature.
i) Spin at 3300 rpm, 4° C. for 45 minutes,
j) Drain off supernatant
k) Count residue in gamma counter
   1.4 Development of Polyclonal Anti-α-MSH Antibody

A high affinity antibody was raised following immunisation with synthetic α-MSH (N-Acetyl-SYSMEHFRWGKPV-NH₂) (purchased from Bachem, AG, Hauptstrasse 144, CH-4416, Bubendorf, Switzerland) conjugated to Keyhole limpet hemacyanin (KLH) according to conventional procedure described in well known literature (Antibodies. A Laboratory manual. E. Harlow & D. Lane. Cold Spring Harbor Laboratory, 1988) to each of 4 rabbits. A total of 8 injections were given at 3-week intervals.

The details are as follows:

1. Four rabbits were immunised with 150 μg α-MSH conjugated to 300 μg KLH with glyceraldehyde per rabbit.
2. Immunisations were carried out by Animal Resource unit, University of Auckland. First immunisation used complete Freunds adjuvant. All other Immunisations (3 weeks apart) used incomplete Freunds adjuvant.
3. One rabbit (KM4) developed antibodies that recognised both α-MSH and desacetyl-α-MSH.
   1.5 Lactoperoxidase Iodination of α-MSH
1. Add 5 μL (2 μg) α-MSH in water to an Eppendorf tube.
2. Add 5 μl Na ¹²⁵l (0.5 μCl) to the α-MSH in Eppendorf tube.
3. Add 47 μL 0.1 M Na Acetate buffer, pH 5.6.
4. Add 10 μL lactoperoxidase (Sigma-Aldrich, MO. USA) freshly diluted in water (2 μg/100 μL).
5. Add 5 μL H₂O₂ freshly diluted 1:7,500 in water.
6. Mix and incubate 5 minutes at room temperature.
7. Repeat steps 5 & 6 two more times.
8. Stop reaction by adding 500 μL PBS and 100 μL transfer buffer (Transfer buffer=RIA Assay buffer with 0.1% Triton X-100 (Sigma-Aldrich, MO, USA) and 0.05% NaN₃.
9. Load mix onto a G2 chromatography column (Pharmacia K9) and elute with Transfer buffer.
10. Collect 1 mL fractions, count 10 μL of each fraction in gamma counter to identify the relevant protein peak.
11. Pool the 3-4 tubes on the descending side of the relevant protein peak.

To test the antisera 5 μg α-MSH was iodinated and purified. The iodinated material was incubated overnight at 4° C. with diluted antiserum and increasing amounts of unlabeled melanocortin peptides. One rabbit developed a high affinity antibody which recognised both α-MSH and desacetyl-α-MSH and not ACTH, γ1, γ2, or γ3-MSH (FIG. 1).

### Example 2:

### Plasma MSH Peptid Content in Normal and Obese Mice

Adult male mice were anaesthetised with halothane and decapitated. Blood was collected into ice cold tubes containing EDTA, The plasma was separated by centrifugation at 4000 rpm for 10 minutes at 4° C. Plasma from 3-4 mice was pooled and mixed, extracted using Sep-Paks, and MSH peptides separated using HPLC and quantitated using RIA Table 1 below shows the MSH data.

TABLE 1
Plasma from 3-4 mice were pooled and assayed
for MSH peptides using HPLC and RIA assays.

| MOUSE | α-MSH | des-α-MSH | α-MSH + des- | des-α-MSH/ |
|---|---|---|---|---|
| TYPE | (pg/ml) | (pg/ml) | α-MSH (pg/ml) | α-MSH |
| A^{VY} yellow | 11.8 | 15.6 | 27.4 | 1.32 |
| male (obese) | | | | |
| A^{VY} black | 19.5 | 16.4 | 35.9 | 0.84 |
| male (lean) | | | | |

The obese mice had a substantially higher des-α-MSH/α-MSH ratio than the lean mice. This was primarily due to a substantially lower level of α-MSH in the obese animals. Within a population this can also be interpreted as having high des-α-MSH in the obese subjects.

### Example 3:

### In Vivo Biological Response of the Hypothalamus to Alpha-MSH and Desacetyl-Alpha-MSH Peptides

Alpha-MSH and desacetyl-α-MSH both couple melanocortin receptors to either adenylyl cyclase or calcium-signalling pathways in vitro. To characterise the signal transduction pathways engaged by α-MSH and desacetyl-α-MSH in vivo, rats received an intracerebroventricular (i.c.v.) injection of either phosphate buffered saline (PBS), α-MSH or desacetyl-α-MSH. Three hours later, food intake was measured and hypothalamic tissues were collected for 2D gel electrophoresis-based proteome analysis.

Intracerebroventricular Injection of Melanocortin Peptides in Adult Rats.

Animals:

Adult male Wistar rats (50-60 days old, 230-260 g at the beginning of the experiment) were maintained in individual cages under controlled temperature (23° C.) and reverse lighting (1000-2200 lights off). Standard laboratory chow (NZ Stockfeed Ltd) and tap water were available ad libitum during the adaptation phase. During this time animals were handled daily to minimize the effects of stress on food intake during experiments. Body weight was measured daily before, and one week after cannulation. Any animal showing signs of illness, such as weight loss, poor grooming, or decreased activity, was removed from the study. All animal procedures undertaken were approved by the Animal Ethics Committee of the University of Auckland.

Cannula Placement:

After 7 days of adaptation, animals were subject to cannula placement surgery under 3% halothane/O₂ anaesthesia. A permanent lateral ventricle infusion cannula (6-mm 21 gauge) was placed on top of the dura at 7.5 mm anterior from stereotaxic zero, 1.5 mm to the right of the mid-sagittal line, and secured to the skull with dental cement. Animals were allowed at least 7 days to recover from surgery before injections.

ICV Infusion of Melanocortin Peptides.

Rats were fasted overnight before the day of experiment. Starvation serves to increase baseline food intake during the initial few hours of testing melanocortin peptide effects on food intake, thereby providing a greater range in which the effect of the anorectic agent α-MSH could be demonstrated.

Under 3% halothane/O₂ anaethesia rats were infused icv through a 12-mm 27-gauge needle, connected to 20-cm length tubing attached to a syringe. Infusions were performed in the early dark phase between 1000 and 1130 hr using motor driven infusion pumps at a rate of 1.0 μl/min over 10 min. Movement of a 0.2 ml air-space introduced between the 0.9% saline solution filling the PE10 tubing-syringe system and the test solution served as an indicator of a successful infusion. At the end of each experiment animals were euthanised by pentobarbital overdose, and cannula placement was confirmed by visual inspection of the cannula tip location within the brain ventricular system.

Proteome Analysis

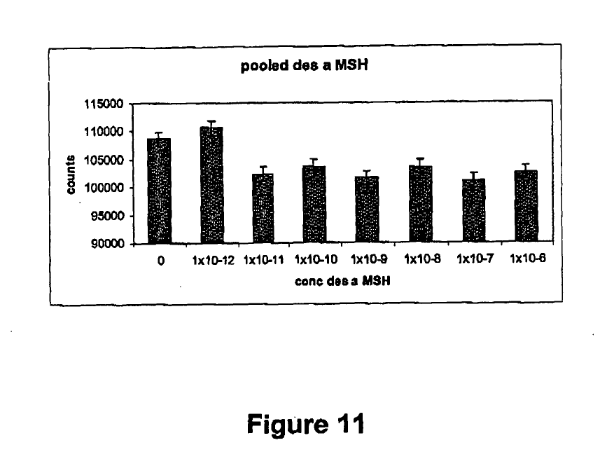

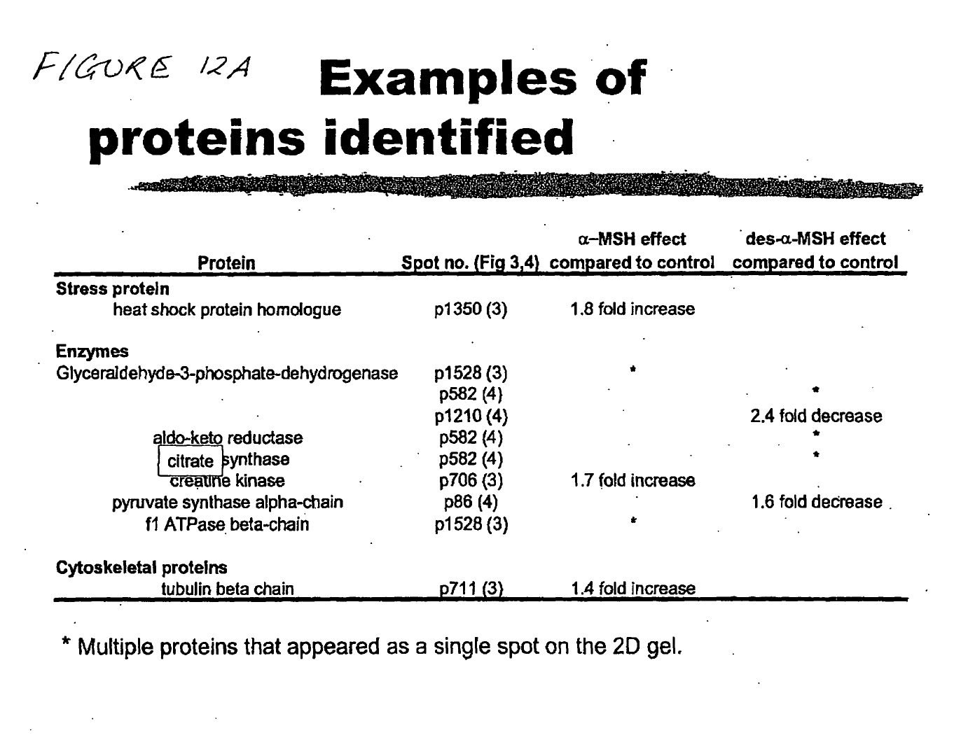

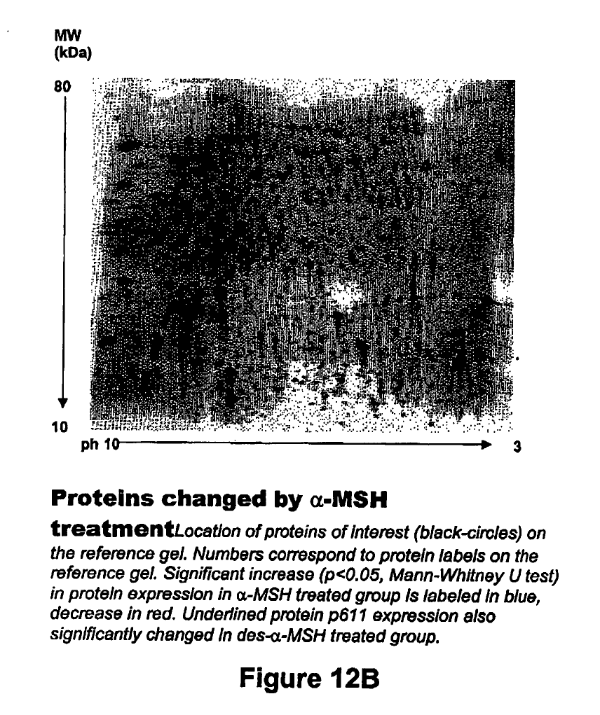

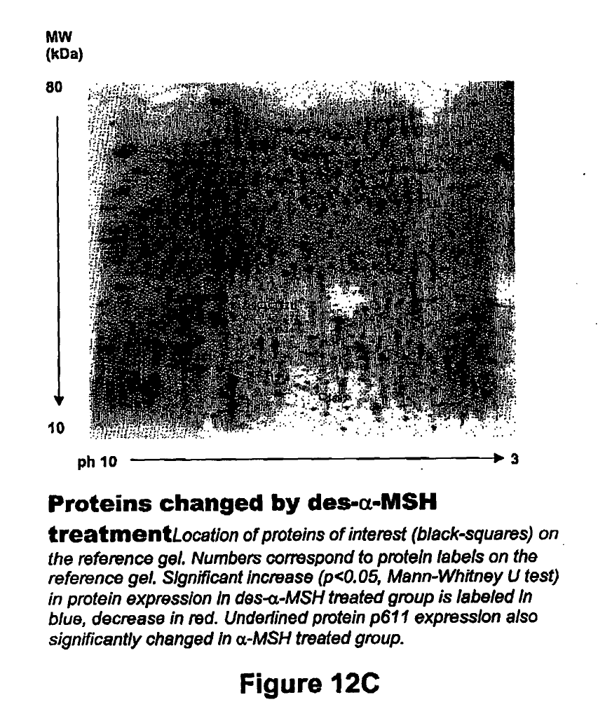

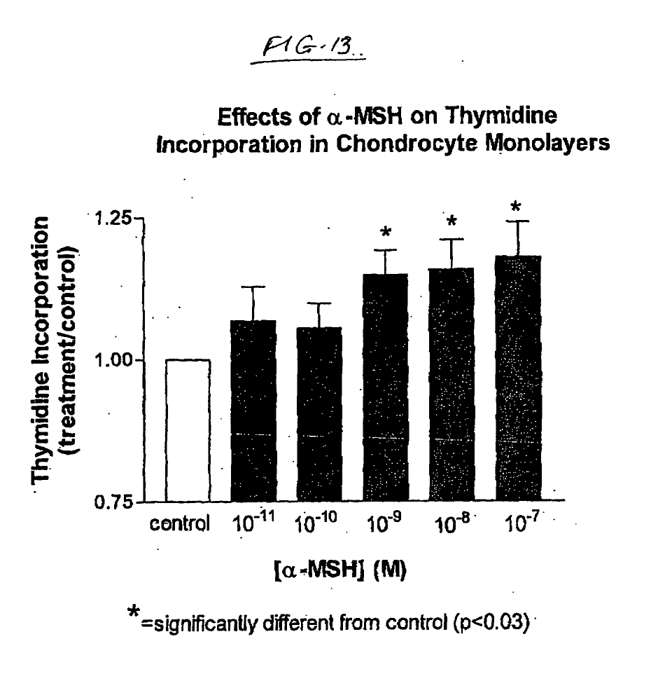

Proteome analysis showed that the expression of 14 proteins were significantly different between PBS and α-MSH, and 20 proteins were significantly different between PBS and desacetyl-α-MSH treated groups (p<0.05, non-parametric/Mann-Whitney U test). Only one of these proteins was common to α-MSH and desacetyl-α-MSH. A combination of Reverse-phase HPLC followed by Edman protein sequencing, and peptide mass fingerprinting technique using MALDI-TOF mass spectrometry were used to identify the proteins of interest. The proteomic data provide a snap-shot of the protein expression patterns in the hypothalamus 3 hours post i.c.v. administration of the melanocortin peptides. The expression of different hypothalamic proteins following administration of either α-MSH or desacetyl-α-MSH supports the hypothesis that these peptides activate different biological responses in vivo by activating different molecular and cellular signalling pathways (FIGS. 11A to 11C).

Tables 1 and 2 represent data from central injection of MSH peptides into brain.

Tables 3 and 4 represent data from a neonatal study, where the two MSH peptides were injected subcutaneously into new-born rats for 14 days, and the changes in hypothalamic proteins assessed with the same method as the above study.

Tables below also show identity of proteins useful as a profile or as markers for the biological response system.

TABLE 1

| | | | | | | |
|---|---|---|---|---|---|---|
| | | | | | | no. datafiles |
| Spot no. | Protein name | Accession no. | Database Nr | Protein coverage | Score | matching |

Proteins significantly changed by α-MSH treatment

| | | | | | | |
|---|---|---|---|---|---|---|
| p428 | Vimentin* | gi/2078001 | 51546 | 2.2 | 2.661# | 1 (2+) |
| p540 | Heat shock 70 kD | gi/13435696 | 70809 | 11.6 | 50.3 | 8 |
| p711 | Similar to tubulin beta polypeptide | gi/13097483 | 33983 | | 2.446# | 1 (2+) |
| p1350 | Similar to heat shock 71 kD* | gi/20853631 | 33053 | 17.9 | 26.3 | 5 |
| | Cofilin 1 | gi/12861068 | 24761 | | 3.5803# | 1 (2+) |
| p1528 | F1-ATPase beta subunit* | gi/203033 | 38729 | 15.4 | 40.4 | 4 |
| | GAPDH | gi/8393418 | 35787 | 8.1 | 18.5 | 4 |
| | Tubulin | gi/13324679 | 49477 | 5 | 10.3 | 2 |
| p1625 | Diazepam binding inhibitor | gi/13837379 | 10028 | 13.6 | 30.3 | 2 |
| | Alpha-enolase | gi/20850814 | 47625 | 4.2 | 38.3 | 3 |

Proteins significantly changed by desacetyl-α-MSH treatment

| | | | | | | |
|---|---|---|---|---|---|---|
| p582 | GAPDH* | gi/8393418 | 35787 | 10.5 | 30.4 | 4 |
| | Aldo-keto reductase family 1* | gi/13591694 | 36464 | 5.5 | 20.3 | 3 |
| | Citrate synthase* | gi/16543177 | 51815 | 8.2 | 10.3 | 3 |
| p1267 | Phosphatidylethanolamine binding protein* | gi/8393910 | 20801 | 25.1 | 10.5 | 3 |
| | Malate dehydrogenase | gi/15100179 | 36442 | 6.6 | 20.3 | 2 |
| p1347 | Cu/Zn superoxide dismutase | gi/1213217 | 15997 | 16.1 | 20 | 2 |
| p1438 | Gamma enolase, neuron-specific | gi/119349 | 47092 | 3.5 | 20.8 | 2 |
| p1521 | Triosephosphate isomerase* | gi/12621074 | 26885 | 10.8 | | 2 |
| | Tubulin beta polypeptide | gi/13097483 | | | 2.581# | 1 (2+) |
| p1548 | Pyruvate kinase 3 | gi/20890302 | 57985 | 5.1 | 40.3 | 2 |
| | Isocitrate dehydrogenase 3 (NAD+) alpha | gi/18250284 | 39639 | 6 | 20.4 | 2 |
| | Phospatidylethanolamine binding protein | gi/8393910 | 20601 | 15 | 30.4 | 2 |
| | GAPDH | gi/20845424 | 35828 | 6.3 | 10.4 | 2 |
| p1687 | Acidic type mitochondrial creatine kinase | gi/125316 | 46981 | 4.8 | 21.1 | 2 |

TABLE 2

| | α-MSH | des-α-MSH |
|---|---|---|
| | effect compared | effect compared |
| Protein | to control | to control |
| Stress protein | | |
| heat shock protein homologue | 2.3 fold increase | |
| (p540) | | |
| heat shock protein homologue | 2.7 fold increase | |
| (p1350) | | |
| Enzymes | | |
| Protein disulfide isomerase | | 1.4 fold decrease |
| (p261) | | |
| glyceraldehyde-3-phosphate- | | 1.4 fold decrease |
| dehydrogenase (p1210) | | |
| creatine kinase (p706) | 2.0 fold increase | |
| triosephosphate isomerase | | 1.4 fold decreasa |
| (p1521) | | |
| gamma-enolase (p1438) | | 1.7 fold decrease |
| Cu/Zn superoxide dismutase | | 1.4 fold decrease |
| (p1347) | | |
| Cytoskeletal proteins | | |
| tubulin beta chain (p711) | 1.6 fold increase | |
| Vimentin (p428) | 1.0 fold increase | |
| Signaling proteins | | |
| phosphatidylethanolamine | | 2.5 fold decrease |
| binding protein (p1267) | | |

TABLE 3

| | | | | | Coverage | |
|---|---|---|---|---|---|---|
| Spot no. | Protein name | Accession no. | Database Nr | Matches | % | Score |
| p537 | dihydropyrimidinase-like 2 | gi/20876560 | 62.3 | 7 | 16.08 | 68.4 |
| p1079 | creatine kinase, mitochondrial 1 | gi/20911541 | 47.0 | 2 | 5.52 | 20.3 |
| p1251 | creatine kinase, brain | gi/6978659 | 42.7 | 5 | 18.37 | 58.4 |
| p1317 | thiol-specific antioxidant protein | gi/16758348 | 24.8 | 10 | 50.44 | 96.8 |
| p1332 | tubulin beta | | | | | |
| p1339 | triosephosphate isomerase | gi/68423 | 26.7 | 8 | 36.15 | 156.7 |
| p1351 | ATP synthase, H+ transporting, | gi/6680748 | 59.8 | 8 | 15.55 | 164.3 |
| | mitochondrial | | | | | |
| p1360 | | | | | | |
| p1362 | spectrin alpha chain, brain, | gi/17380501 | 28.5 | | | 114.3 |
| | fragment | | | | | |
| p13631, | similar to phosphoglycerate kinase | gi/20844750 | 44.6 | 5 | 12.95 | 46.3 |
| | fragment | | | | | |
| p1379 | ATP synthase, H+ transporting, | gi/6680748 | 59.8 | 4 | 4.88 | 60.3 |
| | mitochondrial | | | | | |
| p1381 | hypothetical protein | gi/17389257 | 25.8 | 5 | 20.26 | 70.3 |
| p1414 | gial fibrillary acidic protein (GFAP), | gi/387163 | 46.8 | 1 | 2.98 | 2.807# |
| | fragment | | | | | |
| p1445 | heat shock protein 70 kDa, fragment | | | | | |
| p1454 | triose-phosphate isomerase | gi/68423 | 26.7 | 1 | 5.6 | 3.619# |
| p1458 | similar to prohibitin (B-cell | gi/20912895 | 29.8 | 1 | 3.68 | 2.726# |
| | receptor), fragment | | | | | |
| p-1468 | tubulin alpha3 | gi/6678465 | 50.0 | 1 | 14/? | 3.865# |
| p1520 | similar to tubulin beta polypeptide | gi/13097483 | 34.0 | 5 | 24.83 | 50.3 |
| p1532 | ATP synthase, H+ transporting, | gi/6680748 | 59.8 | 2 | 3.25 | 20.3 |
| | mitochondrial | | | | | |
| p1542 | cofilin 2 | gi/6671746 | 18.7 | 5 | 34.94 | 48.4 |
| | creatine kinase, brain, fragment | gi/6978659 | 42.7 | 3 | 12.34 | 28.3 |
| | lactate dehydrogenase B, fragment | gi/6981146 | 36.6 | 3 | 6.89 | 40.3 |
| p1557 | similar to SH3-containing protein | gi/20823778 | 44.1 | 3 | 7.34 | 36.3 |
| p1558 | tumor necrosis factor | gi/7305585 | 25.9 | 1 | 16/? | 2.382# |
| p1567 | ATP synthase, H+ transporting, | gi/6680748 | 59.8 | 4 | 4.88 | 116.3 |
| | mitochondrial | | | | | |
| p1588 | stathmin, Ser38* | gi/8393696 | 17.3 | 11 | 47 | 188.6 |
| p1610 | stathmin | gi/8393696 | 17.3 | 12 | 55.7 | 228.1 |
| p1690 | spectrin alpha chain, brain, | gi/17380501 | 28.8 | 8 | 3.2 | 114.3 |
| | fragment | | | | | |
| p1754 | tubulin | gi/12846758 | 49.6 | 1 | 4.1 | 6.928# |
| p1757 | unknown protein | gi/1739129 | 27.0 | 2 | 6.98 | 28.3 |
| p1790 | histidine triad nucleotide-binding | gi/20880590 | 13.8 | 5 | 42.9 | 85.7 |
| | protein | | | | | |
| p1827 | glyceraldehyde-3-phosphate | gi/8393418 | 35.8 | 4 | 8.11 | 62.4 |
| | dehydrogenase | | | | | |
| p1854 | cofilin 1, fragment | gi/12861068 | 24.8 | 3 | 8.3 | 50.3 |
| p1936 | creatine kinase, brain | | | | | |

TABLE 4

| Protein Identity | | Protein no. | Level compared to control |
|---|---|---|---|
| Proteins changed by α-MSH treatment: | | | |
| Metabolic enzymes | | | |
| ATP synthase H+ transporting | | p1351 | 2.5 fold increase |
| ATP synthsse H+ transporting | | p1567 | 2.2 fold increase |
| ATP synthase H+ transporting | | p1532 | 2.0 fold increase |
| creatine kinase brain | | p1079 | 1.8 fold increase |
| triosephosphate isomerase | | p1454 | 5.0 fold increase |
| cytoskeleton | | | |
| tubulin alpha | | p1468 | 2.7 fold increase |
| tubulin beta | | p1332 | 1.4 fold increase |
| tubulin beta | | p1520 | 2.4 fold increase |
| tubulin beta | | p1754 | 1.6 fold increase |
| spectrin fragment | | p1690 | 1.8 fold increase |
| glial fibrillary acidic protein | | p1414 | 1.4 fold increase |
| cofilin | | p1854 | 2.5 fold increase |
| signalling | | | |
| prohibitin homologue | | p1458 | 1.7 fold increase |
| stathmin | | p1610 | 2.1 fold increase |
| stress response | | | |
| thiol-specific antioxidant protein | | p1317 | 4.2 fold increase |
| heat shock protein | | p1445 | 2.1 fold increase |
| Unknown function | | | |
| protein kinase C inhibitor | | p1790 | 2.0 fold increase |
| Proteins changed by desacetyl-α-MSH treatment: | | | |
| Metabolic enzymes | | | |
| creatine kinase brain | | p1079 | 1.9 fold increase |
| creatine kinase brain | | p1251 | 2.1 fold increase |
| triosephosphate isomerase | | p1339 | 2.1 fold increase |
| similar to phosphoglycerate kinase | | p1363 | 1.6 fold increase |
| ATP synthase, H+ transporting | | p1379 | 1.7 fold decrease |
| Cytoskeleton | | | |
| spectrin fragment | | p1362 | 2.5 fold decrease |
| cofilin | | p1854 | 1.9 fold increase |
| tubulin beta | | p1520 | 1.6 fold increase |
| Signalling | | | |
| stathmin | | p1610 | 2.6 fold increase |
| stathmin | P*Ser38 | p1588 | 1.7 fold increase |
| prohibitin homologue | | p1458 | 1.7 fold increase |
| Stress response | | | |
| heat shock protein | | p1445 | 2.0 fold increase |
| dihydropyrimidinase-like 2 | | p537 | 2.5 fold decrease |
| Unknown proteins | | | |
| RIKEN cDNA0810011D08 | | p1381 | 3.3 fold increase |
| similar to SH3-containing protein SH3GLB2 | | p1557 | 4.0 fold increase |
| protein kinase C inhibitor | | p1790 | 1.8 fold increase |
| hypothetical protein XP_112457 | | p1936 | 2.2 fold increase |

Measurement of Food Intake:

Following infusion, the cannula was left in place for 1 min, removed, and the animal returned to its cage with fresh pre-weighed food and water. At 3 h post-injection, the pellets and collected food spillage in the cage, were weighed and this weight was subtracted from the initial weight to quantify the amount of food eaten over 3 h.

Statistical Analysis:

The significance of treatment effects was evaluated using one-way ANOVA (Systat10 package)

RESULTS

Alpha-MSH is More Potent than Desacetyl-α-MSH at Inhibiting Food Intake.

Alpha-MSH (10 μg) administered i.c.v to food deprived adult rats just prior to the 12 h dark cycle significantly reduced food intake over 3 h compared to PBS treated control animals (α-MSH, n=7; PBS, n=9; p<0.05). There was a trend for desacetyl-α-MSH (10 μg) to also decrease food intake (n=10) over 3 h, but this was not significantly different from the PBS treated control group of rats.

A 5-fold higher dose of desacetyl-α-MSH (50 μg) did significantly reduce food intake over 3h compared to PBS treated control animals (desacetyl-α-MSH, n=11; PBS, n=11 p<0.05) in a second independent study. In this study α-MSH (10 μg) again significantly inhibited food intake over 3 h compared to PBS treated control animals (α-MSH, n=11; p<0.05).

### Example 4:

### In Vivo Biological Response to the Subcutaneous Administration of Alpha-MSH and Desacetyl-Alpha-MSH Peptides in Rats

The activity of alpha-MSH and desacetyl-alpha-MSH when administered peripherally was measured by subcutaneous administration to postnatal rats for 14 days.

Animals:

Adult female Wistar rats were housed in plastic cages and kept on a 12-h dark/light cycle. Animals received tap water and rat pellets ad libitum and were mated with males of the same strain. Each litter of new-born Wistar rats was culled to 9 pups per mother.

Subcutaneous Injections of Melanocortin Peptides:

Each litter was assigned to a treatment group; vehicle, phosphate buffered saline (PBS), α-MSH (0.3 μg/g body weight/day), or desacetyl-α-MSH (0.3 □g/g body weight/day). PBS or freshly prepared peptide solutions made up freshly in PBS containing 0.1% BSA were injected subcutaneously once per day in a volume of 40 μl for 14 days. Animals were injected on day 14 and 1 h later they were euthanised using sodium pentobarbital.

Measurement of Body and Organ Weights:

Rats were weighed at birth and then every 2 days prior to injection of peptides. Body weights were recorded on day 14 before injection and again when they were euthanised. The following organs were dissected and weighed: brain, heart, kidney, liver, lung, spleen.

Statistical Analysis:

Liner relationships between organ weights and body weights was tested using regression analysis of the organ weights measured against final body weight on day 14. There were significant linear relationships between organ weights and body weights for the following tissues: brain, spleen, heart, kidney and liver. There was no significant regression between lung weight and body weight. For those organs where their weight was linearly correlated to body weight, treatment effects on organ weight changes were analysed using ANCOVA with body weight as the co-variate.

Differences in body weight were analysed using a General Linear Model with repeated measures. Significance was assumed at the P<0.05 level.

Desacetyl-α-MSH Significantly Slowed Body Weight Change in Neonatal Rats.

Three litters of neonatal rats injected daily with desacetyl-α-MSH (0.3 μg/g body weight/day) for their first two weeks of life grew significantly slower than control pups injected daily with PBS (4 litters). In contrast, α-MSH (0.3 μg/g body weight/day) injected daily in neonatal rats (3 litters) had no significant effect compared to control pups injected with PBS. Body weight data obtained from these subcutaneous injections of melanocortin peptides were analysed as a nested within nested design, with the following independent factors: Treatment effects, Litter (Treatment) effects, and Rat (Litter*Treatment) effects. This analysis allowed the separation of sources of variation due to treatment effects, from between litter and between individual rat, differences. Data were analysed using a General Linear Model with repeated measures. Pups treated with desacetyl-α-MSH (n=27) grew significantly slower than either vehicle control (n=36) or alpha-MSH treated pups (n=27) (p,0.05, repeated measures analysis of variance, SAS).

Both α-MSH and desacetyl-α-MSH treated neonatal rats appeared to catch up on body weight from day 12 compared to control PBS treated rats.

Different Effects of Subcutaneously Administered α-MSH and Desacetyl-α-MSH on Organ Weights in Neonatal Rats.

Both α-MSH and Desacetyl-α-MSH (0.3 μg/g body weight/day) administered subcutaneously daily for 14 days to neonatal rats, significantly decreased brain weight compared with control PBS treated animals. Alpha-MSH significantly decreased kidney weight but desacetyl-α-MSH had no significant effect on kidney weight. Desacetyl-α-MSH, however, significantly increased spleen weight but α-MSH had no significant effect on spleen weight.

### Example 5:

### In Vitro Melanocortin Receptor-Mediated Biological Response System

In vitro biological response of primary rat osteoblasts and UMR106.06 rat osteosarcoma cells to melanocortin peptides.

Materials:

The melanocortin peptides, ACTH₁₋₂₄, desacetyl-α-MSH and α-MSH were purchased from Bachem California (CA, USA). The production of recombinant mouse agouti protein has previously been described (Willard, 1995 #760). [³H] Methyl thymidine was purchased from Amersham Life Science (Buckinghamshire, U.K.).

Cells:

Rat osteosarcoma UMR106.06 cells were grown in Dulbecco's modified Eagle's Medium (DMEM) (GIBCO BRL, Rockville, Md.) supplemented with 10% fetal calf serum (FCS) (in Vitrogen, Auckland, NZ) and 50 U/ml penicillin plus 50 μ/ml streptomycin. Cells were maintained at 37° C. in 5% CO₂ and passaged every week.

Primary rat osteoblasts were isolated from 20 day fetal rat calvariae. (The use of animals for these studies was approved by the Auckland Animal Ethics Committee.) Calvariae were excised and the frontal and parietal bones, free of suture and periosteal tissue, were collected and sequentially digested using collagenase as previously described (Comish J, Callon K E, Lin C Q X. Xiao C L, Mulvey T B, Cooper G J S, Reid I R Trifluoroacetate, a contaminant in puritied proteins, inhibits proliferation of osteoblasts and chondrocytes. Amer J Physiol Endocrinol Metab 277: E779-E783, 1999). Primary rat osteoblasts were grown in DMEM supplemented with 10% FCS. 50 U/ml penicillin and 50 μg/ml streptomycin. After 48 hour, the medium was changed to MEM. Confluence was reached within 5-6 days, at which time the cells were subcultured into 10 cm culture plates for RNA preparation or 24 well plates for proliferation assays.

Preparation of mRNA

Total RNA was extracted from adult rat brain, skin, UMR106.06, or primary rat osteoblast cells using the guanidinium thlocyanate method (Chirgwin, 1979 #129). Poly (A)⁺ mRNA was purified from the total RNA using the PolyATract mRNA Isolation System (Promega, Madison, Wis.).

Northern Blot Analysis

Primary rat osteoblast poly (A)⁺ (5 μg) and rat brain poly (A)⁺ were size separated alongside lamda EcoRI/HindIII markers by electrophoresis on a 2.2M formaldehyde-1.2% agarose gel, transferred to a Magnacharge Nylon membrane (MSI, Westborough, Mass.), and hybridised with a rat specific MC4-R gene DNA fragment spanning transmembrane domains III and VII (Mountjoy, 1994 #656). Hybridisation conditions were 50% formamide, 1 mM NaCl, 50 mM Tris-HCl (pH 7.5), sodium pyrophosphate (0.1%), SDS (0.2%), salmon sperm DNA (100 μg/ml), 10× Denhardt's and 10% dextran sulfate at 42° C. for 18 h. A digital image of MC4-R transcripts was obtained after 10 days exposure with a phosphoscreen by using the Storm imaging system scanner (Molecular Dynamics).

PCR Amplification of Reverse Transcribed mRNA (RT-PCR)

Poly (A)⁺ mRNA was DNase treated twice using 10 U RQ1 RNase-free DNase (Promega Corp., Madison, Wis.) per mg poly (A)⁺ mRNA for 30 min at 37° C. each time. First strand cDNA was synthesised using 200 U SuperScript II RNaseH⁻ reverse transcriptase (GIBCO BRL, Rockville, Md.) and oligo (dT)₁₂₋₁₈ (Pharmacia Biotech AB, Uppsala, Sweden) at 42° C. for 1 h in a final volume of 20 μl. To test for DNA contamination of the RNA, a reaction was carried out with 1 □g poly (A)⁺ mRNA and all the reagents but no reverse transcriptase (control reaction). The cDNA and control reaction (2 μl) were used as templates for PCR with rat melanocortin receptor specific oligonucleotides described in Table 1. The PCR conditions were 94° C. for 3 min, 40 cycles of 94° C. for 40 sec, annealing for 40 sec, and 72° C. for 1 min, followed by 72° C. for 10 min. The amplified cDNA products were separated on a 1.2% agarose gel alongside a EcoRI-HindIII-digested lambda DNA ladder and stained with ethidium bromide.

Ribonuclease Protection Assay

The cDNA templates used to synthesise the antisense rMC4-R and rMC1-R riboprobes were generated from 562 and 270 bp respectively, nucleotide DNA fragment spanning transmembrane I to VII and III to VI domains subcloned into pBKS (Stratagene). These recombinant DNA templates were linearised with EcoRI and Sall transcribed with [α-³²P]UTP (Amersham Life Science (Buckinghamshire. U.K.) using T7 RNA polymerase to generate ³²P-labeled cRNA probes. Rat brain or skin, UMR106.06, and primary rat osteoblast poly (A)⁺ mRNA (10 μg) were treated with 2 U RNase-free DNasel (Boehringer Mannheim, Indianapolis, Ind.) at 37° C. for 50 min and the RNA was precipitated. The RNA pellet was resuspended in 20 μl hybridization buffer (80% formamide, 40 mM PIPES pH 6.4, 400 mM NaCl, 1 mM EDTA) with 5×10⁵ cpm of ³²P-labeled riboprobe, denatured at 85° C. for 5 min and hybridized at 45° C. overnight, The hybridised RNA was digested with 40 μg RNase A and 50U RNase T1 at 37° C. for 30 min. The protected RNA fragments were analyzed on a 6% denaturating polyacrylamide gel alongside a ³²P-labeled 123-bp DNA ladder (10⁵ cpm). A digital image of ³²P-labeled fragments was obtained using a Storm imaging system.

In Situ Hybridisation

Neonatal mouse calvariae, tibial and femoral bone were collected from 1-2 and 6 day old Swiss mice that had been euthanised by cervical dislocation while under halothane anesthesia (approved by the Auckland Animal Ethics Committee). The bones were dissected free of adherent soft tissues and fixed in 4% paraformaldehyde for 24 h at 4° C. prior to decalcification (15% EDTA, 4% paraformaldehyde) for 72 h at 4° C. They were then transferred to 10% sucrose, 4% paraformaldehyde overnight at 4° C. before being embedded in OCT and stored frozen at −80° C. Five series of 20 μM of either cross sectional or longitudinal sections were cut on the cryostat and mounted onto polysine coated microscope slides (Biolab Scientific, NZ) and in situ hybridisation performed as previously described (Mountjoy K G, Mortrud M T, Low M J, simerly R B, Cone R D Localization of the melanocortin-4 receptor (MC4-R) in neuroendocrine and autonomic control circuits in the brain. Mol Endocrinol 8: 1298-1308, 1994). Bone sections were hybridised with ³³P labelled cRNA antisense rat MC4-R (628bp). Sections were hybridised in 65% formamide in 0.26M NaCl, 1.3× Denhardt's, 13 mM Tris-HCl pH 8.0, 1.3 mMEDTA, 13% dextran sulphate at 60-65° C. for 18 hours. Sections were washed and coated with emulsion for autoradiography. Following the developing of these slides, the sections were stained with haematoxylin and eosin and then photographed under darkfield on a Leica Microscope (Leitz DMRBE). One series of sections from each case was not subjected to in situ hybridization but was counterstained with haematoxylin and eosin and used for the identification of structures and bone cell type.

Primary Rat Osteoblasts Proliferation Assays

Primary rat osteoblasts were subcultured into 24 well plates at a density of 5×10⁴ cells/ml/well in MEM, 5% FCS for 24 hours. Cells were growth arrested in MEM, 0.1% bovine serum albumin (BSA) for 24 hour and then fresh media and experimental compounds were added for a further 24 hours. Cells were pulsed with [³H]thymidine (0.50□Ci/well) 2 hours before the end of the experimental incubation. The experiment was terminated and both cell numbers and thymidine incorporation were assessed. Cell numbers were analysed by detaching cells from the wells by exposure to trypsin/EDTA (.05%/0.53 mM) for approximately 5 minutes at 37° C. Counting was performed in a hemocytometer chamber. Results are expressed per well. [³H]Thymidine incorporation was analysed by washing the cells in MEM followed by the addition of 10% trichloroacetic acid. The precipitate was washed twice with ethnol:ether (3:1) and the wells desiccated at room temperature. The residue was dissolved in 2M KOH at 55° C. for 30 minutes, neutralized with 1M HCl, and an aliquot counted for radioactivity. Results are expressed as dpm per well. Each experiment was performed at least three times using experimental groups consisting of at least six wells.

Statisitics

Data are presented as mean±SEM. The significance of differences between groups was determined using Student's t tests for unpaired data and a 5% significance level. The comparisons to be made in each experiment were specified a priori, so no adjustment for multiple comparisons was necessary. Where several experiments have been shown in one figure, the data are expressed as the ratio of results in treatment groups to those in the control group and the ‘P’ values shown were calculated using the data from the individual experiments, before the data were pooled.

Results

MC4-R mRNA is Expressed in UMR106.06 and Primary Rat Osteoblast Cells.

Four different methods confirmed expression of MC4-R mRNA in UMR106.06 and rat primary osteoblast cells. First, RT-PCR, using rat specific MC4-R oligonucleotides amplified the correct size DNA fragment from poly A⁺ mRNA and not from genomic DNA. Second, Northern blot analysis of rat primary osteoblast poly (A⁺) mRNA (5 μg) showed a broad band of MC4-R mRNA transcripts between 2.0 and 2.6 kb, the same size as seen in rat brain, albeit of much lower abundance than in brain. Third, RPA's confirmed MC4-R mRNA expression in UMR106.06 and primary rat osteoblast cells. Finally, we used in situ hybridisation to localise MC4-R mRNA expression in the periosteum of 1-2 and 6 day old Swiss mouse calvariae, tibia, and femoral bones.

MC2-R and MC5-R mRNA are Expressed in UMR106.06 and Rat Primary Osteoblast Cells.

RT-PCR, using rat specific MC2-R and MC5-R oligonucleotides amplified correct size DNA fragments from 1 μg UMR106.06 and 1 μg primary rat osteoblast cell poly A⁺ mRNA, but not from genomic DNA.

Alpha-MSH, but not Desacetyl-α-MSH nor ACTH₁₋₂₄, Stimulates Proliferation of Primary Rat Osteoblasts.

Alpha-MSH (10⁻⁹-10⁻⁷ M) significantly increased thymidine incorporation into growth arrested primary rat osteoblasts. Over a similar range of concentrations alpha-MSH also increased osteoblasts cell numbers. Desacetyl-α-MSH (10⁻⁷ M) and ACTH₁₋₂₄ (10⁻⁷ M) did not stimulate thymidine incorporation or cells numbers in growth arrested rat primary osteoblasts.

Desacetyl-α-MSH and ACTH₁₋₂₄ Antagonise α-MSH Stimulated Proliferation of primary Rat osteoblasts.

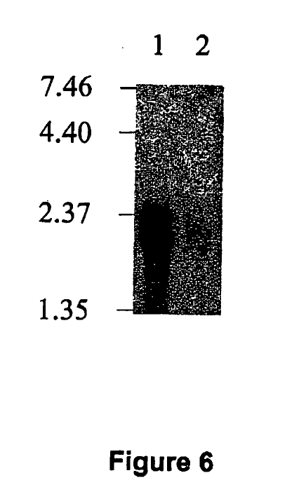

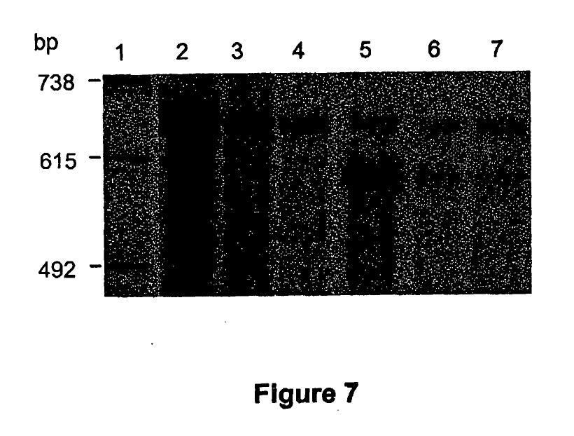

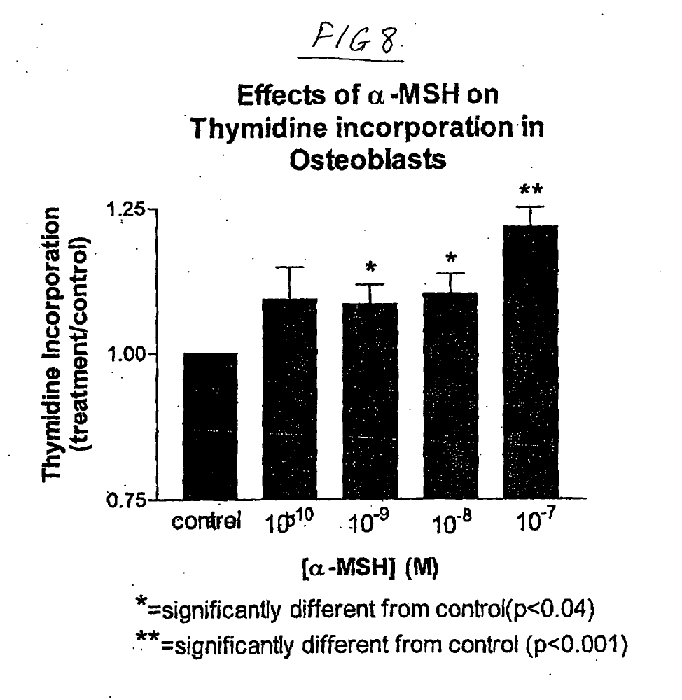

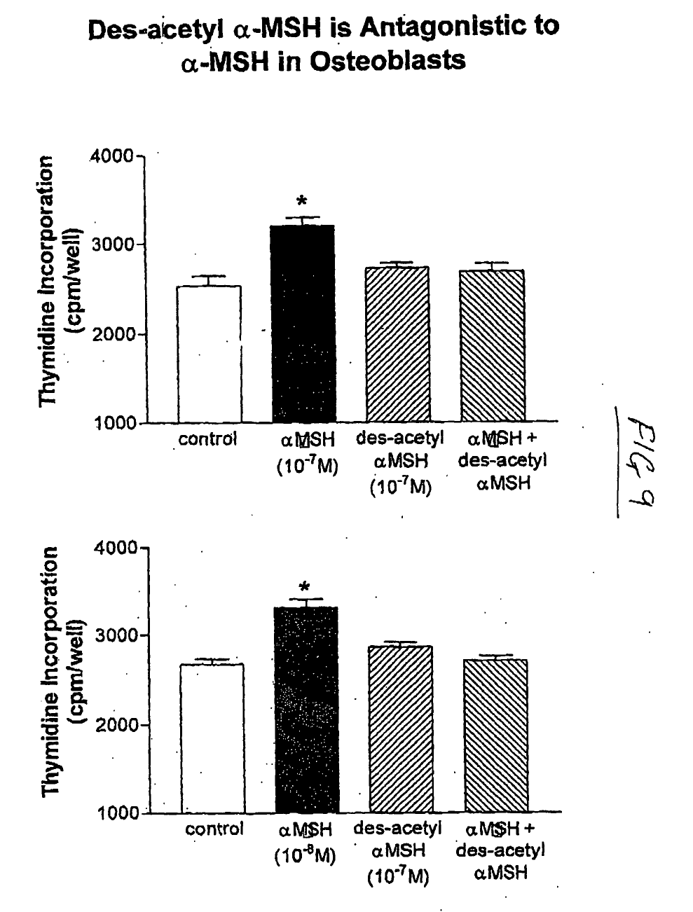

Desacetyl-α-MSH (10⁻⁷M) inhibited two doses of α-MSH (10⁻⁸M and 10⁻⁷M) from stimulating [³H] thymidine uptake into growth arrested rat primary osteoblasts (FIG. 6a). ACTH₁₋₂₄ (10⁻⁷ M) inhibited two doses of α-MSH (10⁻⁸ M and 10⁻⁷M) from stimulating [³H] thymidine uptake into growth arrested rat primary osteoblasts.

Discussion

The MC4-R is likely to play a direct role in bone metabolism since its mRNA is expressed in a rat osteosarcoma cell line as well as in primary rat osteoblasts. The full length mRNA transcript for MC4-R expressed in primary rat osteoblasts is between 2 and 2.6 kb, the correct size for producing a functional protein in these cells. Expression of MC4-R mRNA is, however, much less abundant in osteoblasts than in rat brain, where MC4-R mRNA expression is already considered to be very low compared with many other genes. The MC4-R is not the only melanocortin receptor expressed in osteoblasts since we also observed MC2-R and MC5-R mRNA expressed in very low abundance in primary rat osteoblasts. Despite the low abundance of melanocortin receptors, melanocortin peptides have significant biological effects on osteoblast cell proliferation.

Alpha-MSH (10⁻⁹-10⁻⁷M) significantly stimulated both thymidine uptake and increased cell number in primary rat osteoblasts. The EC₅₀'s for α-MSH coupling mouse MC4-R and MC5-R to adenylyl cyclase or mobilisation of intracellular calcium are in the 10⁻⁹M range, and therefore the α-MSH-stimulated osteoblast proliferation could be mediated by either MC4-R or MC5-R, or both. Alpha-MSH does not stimulate the MC2-R. Surprisingly, ACTH₁₋₂₄ had no significant effect on osteoblast proliferation and yet ACTH₁₋₂₄ functionally couples MC2-R, MC4-R, and MC5-R to adenylyl cyclase when these receptors are overexpressed in various cell lines. Desacetyl-α-MSH (10⁻⁷M and 10⁻⁸M) also had no significant effect on osteoblast proliferation in two out of three experiments, and yet the EC₅₀'s for desacetyl-α-MSH coupling MC4-R and MC5-R to intracellular signaling pathways when these receptors are overexpressed in heterologous cells are similar to those for α-MSH.

To further understand the significance of MC4-R mRNA expression in osteoblasts we attempted to antagonise the α-MSH stimulated osteoblast proliferation. Agouti protein is an antagonist of melanocortin peptides coupling MC1-R, MC2-R, and MC4-R. However, in our study agouti protein alone (10⁻⁹M-10⁻⁷M) significantly stimulated thymidine incorporation in primary rat osteoblasts and did not antagonism α-MSH stimulated osteoblast proliferation. Furthermore, agouti protein stimulated-thymidine incorporation was not additive with α-MSH stimulated-thymidine incorporation, suggesting that agouti protein and α-MSH may be having their effects through the same melanocortin receptor and signal transduction pathway.

We were unable to distinguish between the three subtypes of melanocortin receptors expressed in osteoblasts based on biological activities of melanocortin receptor agonists, and the MC2-R/MC4-R antagonist, agouti protein. This is not the first time however, that the biological activities of melanocortin receptor ligands on endogenous melanocortin receptors differ from their biological potencies on cloned melanocortin receptors overexpressed in heterologous cells. First, α-MSH and desacetyl-α-MSH are potent agonists of the cloned MC1-R overexpressed in heterologous cell lines, but only α-MSH potently stimulates pigmentation in rodent skin. Second, NDP-MSH is a potent agonist of cloned MC5-R overexpressed in heterologous cell lines, but it is a potent antagonist of α-MSH activation of adenylyl cyclase in 3T3-L1 adipocytes. It is possible that the very low expression of endogenous melanocortin receptors in primary osteoblasts, melanocytes, and 3T3-L1 adipocytes contributes to the differences in melanocortin potencies in these cells compared with overexpressed cloned melanocortin receptors. Additionally, 3T3-L1 adipocytes, like primary osteoblasts, express more than one melanocortin receptor subtype. It is likely therefore, that heterodimeric receptors are formed and these could have different pharmacological profiles from homodimers formed when each cloned melanocortin receptor is overexpressed alone.

Without wishing to be bound by any particular mechanism of action it is proposed that osteoblasts are a model system for understanding interactions between melanocortin receptor ligands and melanocortin receptors, and this model system more closely resembles in vivo responses to melanocortin peptides compared with overexpressing only one melanocortin receptor in an heterologous cell. It has been shown that while desacetyl-α-MSH or ACTH₁₋₂₄ alone had no agonist effects on osteoblast proliferation, they were both capable of antagonising α-MSH stimulated osteoblast proliferation. This study is the first to report the ACTH₁₋₂₄ antagonism of α-MSH. Desacetyl-α-MSH antagonises α-MSH stimulated mammotrope recruiting activity in anterior pituitary cell cultures (Ellerkmann E, Kineman R D, Porter T E, Frawley L S Des-acetylated variants of α-melanocyte-stimulating hormone and β-endorphin can antagnize the mammotrope-recruiting activity of their acetylated forms. J Endocrinology 139: 295-300, 1993) and antagonises α-MSH activity on Anolis melanophore (McCormack A M, Carter R J, thody A J, Shuster S Des-acetyl MSH and γ-MSH act as partial agonists to a-MSH on the Anolis melanophore. Peptides 3:13-16, 1981).

Low level endogenous expression of three melanocortin receptor subtypes in osteoblast cells provides a model system (FIG. 8) for exploring interactions between melanocortin receptor ligands and melanocortin receptors that will more accurately reflect the in vivo actions of melanocortin peptides, agouti, and agouti gene related peptide. In osteoblasts, and probably many cell types expressing low levels of endogenous melanocortin receptors, them is the likelihood of melanocortin receptor homo- and heterodimers, and cross talk between different melanocortin receptors. These interactions would provide diversity and specificity for melanocortin peptide signalling that would not be available when a single melanocortin receptor subtype is overexpressed in heterologous cells.

It is evident that a variety of cell types and tissues may express melanocortin receptors. In addition to those described above, any such cells or tissues would be appropriate candidates as a biological response system, according to the invention described herein. Examples of cell lines that could be utilised in a similar manner as described above include the GT1-7 mouse hypothalamic cell line, 3T3-L1 adipocytes, melanocytes, L6 myocytes, B16 melanoma cells, and anterior pituitary cell cultures.

Genetically engineered, or heterologous cell lines that stably express a single or a combination of melanocortin peptides are also good candidates as in vitro cellular biological response systems. A panel of such cell lines, each expressing a different melanocortin receptor may comprise a biological response system. Alternatively, co-cultures of two or more heterologous cell lines, each expressing different melanocortin receptors may comprise a biological response system.

### Example 6:

### Biological Response by UMR106.06 Rat Osteosarcoma Cell Line.

Incorporation of Tritiated Thymidine into DNA

UMR106 cells are plated at at 1×10⁵ cells/well in a 24 well plate using 10% FCS, DMEM media. 24 hours later the medium is changed to serum free medium containing 0.1% BSA. Following a 24 hour incubation period, the medium is changed again to serum free media containing 0.1% BSA and increasing concentrations of melanocortin peptides. The cells are then incubated for 22 hours. Following this period of incubation {methyl-3H} thymidine [0.5 μCi in 25 μl/well] is added and left for 2 hours at 37° C. (use 0.5 μl of 1 μCi/μl tritiated thymidine into 24.5 μl 0.1% BSA, DMEM for each well). The experiment is terminated by washing the cells with 1 ml cold PBS and then add 1 ml cold 5% TCA.

The plates are then left at 4° C. (on ice) for 15 minutes and then washed 3× with 1 ml cold 5% TCA and twice with 1 ml absolute ethanol. The monolayers are air dried and cells dissolved in 1 ml 0.3N NaOH by heating at 37° C. for 1 hour. 200 μl of 1.5N HCL is then added to each well and then the contents of each well is transfered to individual 20 ml glass scintillation vials. 7 mls of scintillation fluid is added and mixed well. The samples are counted for 5 minutes.

Results

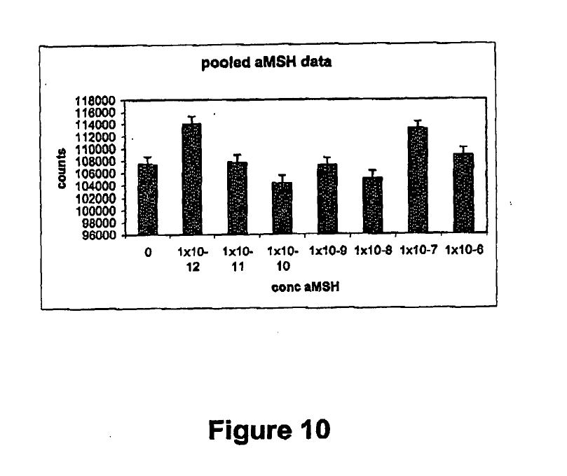

FIGS. 9 and 10 show the proliferation response resulting from the treatment of UMR106.06 rat osteosarcoma cells with varying concentrations of alpha-MSH or desacetyl-alpha-MSH.

This example is illustrative of the usefulness of a permanent cell line that can be used as an in vitro biological response system. Of course, it will be understood that a proliferative response is only one of many response parameters that may be utilized as a response profile.

### Example 7:

### Use of the In Vitro Biological Response System to Screen for Compounds that Act as Agonists or Antagonists of Melanocortin Receptors

An in vitro biological response system may be utilised to screen for compounds that act as agonists or antagonists of melanocortin receptors. Such a biological response system could also be utilised to screen for compounds that are useful in the treatment of subjects suffering from obesity or an imbalance in energy homeostasis or disturbance in feeding/weight gain patterns.

The screening process involves treating the cells of the biological response system having the appropriate combination of receptors with test compounds and then measuring the response parameters, either by mass spectrometry or by gene expression array or by other available techniques which are able to assess the required response parameters. The compound that produces the desired response profile is a compound which may be useful in the treatment of obesity or imbalances in energy homeostasis and/or disturbances in feeding/weight gain patterns. The biological response system will also enable the selection of compounds that are able to block the undesirable effects of environmental and nutritional factors that cause obesity or imbalances in energy homeostasis and/or disturbances in feeding/weight gain patterns.

The profile generated by compounds that produce a desired response in an in vitro biological response system may then be compared with the profile that is generated from the administration of the compound to an in vivo biological response system.

### Example 8:

### In Vitro Biological Response of 3T3 L1 Adipocytes to Melanocortin Peptides

(i) Culturing Murine 3T3 L1 Cells

Culturing and passaging cells based on methods described in references Norman D et al Mol Cell Endocrinol 200: 99-109, 2003; Hasegawa N et al Phytother Res 16: S91-S92, 2002: Student AK et al J Biol Chem. 255: 4745-4750, 1980; and Ross S E et al Mol Cell Biology 19: 8433-8441, 1999, all of which are incorporated herein in their entirety by reference.

Reagents

1.1 Growth Medium;
a-MEM culture medium: powder from GibcoBRL, prepared in advance and stored in volumes of 225 ml in sterile culture bottle at 2-8° C.
Fetal Bovine Serum (FBS): GibcoBRL, sterile heat-inactivated serum stored in 25 ml aliquots in 50 ml tubes in −20° C. freezer.

To heat inactivate place serum in water bath set to 50° C. for 1 hour.
Penicillin/Streptomycin (P/S): (GibcoBRL 15070-063, 100 U/ml, 100 mg/ml,) stored in sterile aliquots in −20° C. freezer.

1.2 Reagents for passaging pre-adipocytes Growth medium
Trypsin: (GibcoBRL 25300-024, 100 ml) stored in sterile 15 ml tube aliquots in −20° C. freezer.
Versene: (GibcoBRL 15040-066, 1:5000, 100 ml) stored in sterile bottle at 2-8° C. Versene is EDTA, a calcium chelator used to remove calcium, which helps cells attach to plate.

4.1 Plating cells:
Transfer cell suspension from cryotube to a 5 ml medium tube, centrifuge at room temperature (20-22° C.) at 960 rpm for 5 min, aspirate supernatant, leaving approximately 2 mm supematant above pellet so that cell pellet is not disturbed.
Add 10 ml medium and resuspend with 10 ml pipette, gently drawing up medium and releasing along side of tube approximately 10 times to disperse cells.
Transfer cell suspension to labelled petri dish (tech name, date, cell ID) and examine under microscope (10× objective), checking that there are no cell clumps. Place in incubator at 37° C. and 5% CO₂.
   Passaging Pre-adipocytes to Increase Cell Number Detaching Cells from Plate;

5.1 Pre-adipocytes are ready for passaging every 4-5 days (cells are not confluent and generally only 5-10% differentiated).
Transfer 5 ml growth medium into 15 ml tube.
Remove culture plate of 3T3 L1 cells from incubator and place in hood. Aspirate medium.
Add 2 ml Versene to plate, allowing it to run down inside wall of plate to avoid dislodging cells. Gently swirl to run over whole bottom of plate, then aspirate immediately.
Add 2 ml trypsin over whole bottom of plate. Tap bottom of plate, place in incubator for ˜1 minute, check under microscope that cells are dislodged and not clumpy.
Transfer cells to tube with 5 ml medium and centrifuge at approximately 21° C. for 5 minutes at 960 rpm.

5.2 Passaging cells:
While cells are spinning, place 9 ml fresh medium into each labelled culture plate.
After spin, aspirate cell supematant (down to ˜1 mm from pellet).
Add 10 ml medium and mix to resuspend with several up/down strokes (˜10).
Transfer 1 ml into each plate.
Examine under microscope to check cells and for absence of cell clumps.
Place in incubator, 37° C. and 5% CO₂.
Discard remainder of cells in sealed tube in biohazard bag.
   Oil Red O Staining of Adipocytes

Oil Red O staining is used to determine differentiation efficiency of adipocyte cell lines such as 3T3 L1 cells by staining intracytoplasmic lipid accumulation. This method is broadly based on methods published earlier (Norman D et al Mol Cell Endocrinol 200: 99-109, 2003; Ross S E et al Mol Cell Biology 19: 8433-8441, 1999; Zhang H H et al J Endocrinol 164: 119-128, 2000, incorporated herein intheir entirety by reference).

Materials and Preparation of Reagents

Isopropanol
100% isopropanol
60% isopropanol=60 mL isopropanol+40 mL mille Q H₂O
50% isopropanol=50 mL isopropanol+50 mL mille Q H₂O

Oil Red O Stain
Use at 0.3% in 60% isopropanol
0.3% stain=300 mg Oil Red O+100 mL 60% isopropanol Filter before use.

Phosphate Buffered Saline (PBS) sterile for cell culture, pH 7.4
8 g NaCl+0.2 g KCl+1.44 g Na₂HPO₄+0.24 g KH₂PO₄.
Dissolve in ˜800 ml milli Q water. Adjust pH to 7.4 with 1N HCl.
Bring volume up to 1 L and autoclave.

4% paraformaldehyde, pH 7.4
4%=4 g paraformaldehyde+100 mL PBS
Dissolve by adding 1 pellet NaOH while mixing on heated mixer (˜50° C.).
Adjust pH to 7.4 with 1N HCl.
   Staining Cells

Use the same volume for each reagent, which is determined by plate/well size as Table 1.

Aspirate cell medium and rinse 2× with PBS.

Fix for 1 hour in 4% paraformaldehyde at 4° C. (place in fridge or cold room).

Aspirate paraformaldehyde and rinse 2× with PBS.

Stain with Oil Red O for 20 minutes, leave plate in hood.

Aspirate stain and rinse 2× with water and 1× with 50% isopropanol.

Check staining of cells under microscope.

Elute stained lipids with 100% isopropanol. Check elution efficiency under microscope.

Measure absorbance at 510 nm on spectrophotometer.

TABLE 1
Volume of Reagents Used for Oil Red O Staining

| | Diameter of | Area of | Volume of |
|---|---|---|---|
| | well/plate | well/plate | Reagents |
| Plate | (mm) | (mm²) | (mL) |
| 12-well dish | 20 | 314 | 0.5 |
| 6-well dish | 35 | 962 | 2 |
| Culture plate | 100 | 7854 | 10 |

Differentiation of 3T3 L1 Cells with Indomethacin
Growing Cells and Inducing Differentiation

Differentiation induction with indomethacin bsed on Norman et al (Norman D et l Mol Cell Endocrinol 200: 99-109, 2003). Details of preparation of αMEM growth medium (containing 10% FBS and pen/strep), retrieving and plating cryopreserved 3T3 L1 cells from liquid nitrogen are detailed above.

Passage cells when nearly confluent, in 4-5 days, by splitting 1/10 in new plates and feed every 2 days.

To induce differentiation, 48 hours after cells are confluent add differentiation medium as follows (DAY 0):

Prepare differentiation medium as in 2.0.

Aspirate growth medium from plate.

Add differentiation medium to plate and return to incubator, 37° C. and 5% CO₂. Volume depends on size of well or plate. Use 2 ml/well in 6-well plate or 10 ml/culture plate.

After 48 hours differentiation (DAY 2), aspirate differentiation medium and add growth medium supplemented with 5 ug/mL insulin. Change medium every 2 days.

Perform experiments on DAY 12-14, or later if desired.

Preparation of Indomethacin Differentiation Medium

Indomethacin: (Sigma I 7378, MW=357.8). Use at a final concentration of 125 uM. On day of use dissolve 15 mg/ml in absolute ethanol. A final concentration of 125 uM indomethacin requires 44.725 ug/ml growth medium or 4472.5 ug/100 ml.
1 M=357.8 g/L=357.8 mg/ml
1 mM=357.8 ug/ml
1 uM=0.3578 ug/ml
125 uM=0.3578×125=44.725 ug/ml
For 100 ml medium, use 100×0.044725 mg/ml=4.47 mg.

4.47 mg=298 ul of 15 mg/ml solution (4.4725/15=298 ul).

Insulin (bovine): (Sigma I 6563, MW=5733.5). Use at a final concentration of 5 ug/ml. Prepare a 1 mg/ml solution (store unused solution at −20° C.). For 100 ml medium, use 100×0.005 mg/ml=0.5 mg, which is 0.5 ml of 1 mg/ml.

Calculate volume of differentiation medium required (as in 1.3.3). For 100 ml growth medium add:
298 ul of 15 mg/ml indomethasin solution
500 ul of 1 mg/ml insulin solution.

Mix by swirling.

Stimulation of 3T3 L1 Adipocytes with Melanocortin Peptides

1.0 Methodology:

Method according to Norman D et al (2003) Mol Cell Encrinol 200, p 99-109 was used. This publication is incorporated in its entirety herein by reference.

2.0 Introduction and Overview

2.1 The objective of this study was to determine the effects of a-MSH and desacetyl a-MSH on leptin and triglyceride production in murine 3T3 L1 adipocytes.

2.2 Pre-adipocytes were seeded in 6-well plates and 2 days post confluence (Day 2) were differentiated with 125 mM indomethacin+5 ug/mL insulin (described in previous documents).

2.3 On Day 13 adipocytes were stimulated with 4 doses each of a-MSH and desacetyl a-MSH (or no peptide added) for 4 hours.

2.4 Medium was removed from the wells and leptin and triglyceride levels measured.

2.5 Intracytoplasmic lipid accumulation was measured by staining with Oil red O.

3.0 Reagents

3.1 a-MEM growth medium

3.2 Bovine Serum Albumin (BSA)

3.3 a-Melanocortin Stimulating Hormone (a-MSH), MW 1665

3.4 desacetyl a-Melanocortin Stimulating Hormone (da-MSH), MW 1623

3.5 Phosphate Buffered Saline (PBS), pH 7.4

3.6 Isobutylmethylxanthine (IBMX) Sigma I 7378, MW=222.2.

4.0 Preparation of Reagents

4.1 Medium=a-MEM+0.5% BSA (100 mL a-MEM+0.5 g BSA)

4.2 Doses of a-MSH and da-MSH (stocks in −80° C. freezer=1 ug/ul), using MW of a-MSH.
1 M=1665 g/L=1665 mg/mL
1 mM=1.665 mg/mL
1 uM=1.665 ug/mL
1.665 ug/mL=3.3 ug/2 mL in each well
1/10 dilution of 1 ug/ul (stock)=0.1 ug/ul. 33 ul=3.3 ug.
Prepare 1/10 dilution of freezer stock (1 ug/ul) to make 0.1 ug/ul, using a-MEM+0.5% BSA as diluent.
Make 3 serial dilutions of 1/10 to add 33 ul to each well in 6-well plate.

Doses are in triplicate wells, so require 3×33=99 ul for each dose.

| | | |
|---|---|---|
| Final Dose | | |
| when adding | | |
| 33 ul/well | Stock | Dilution |

| | | | |
|---|---|---|---|
| 1 | uM | A | 20 ul freezer stock + 180 ul medium |
| 100 | nM | B | 20 ul stock A + 180 ul medium |
| 10 | nM | C | 20 ul stock B + 180 ul medium |
| 1 | nM | D | 20 ul stock C + 180 ul medium |

4.3 1 mM IBMX (final concentration)=0.2222 mg/mL. Immediately prior to use on Day 13, prepare 30 mg/6 mL solution in sterile PBS as in “Differentiation with Dexamethasone and IBMX” document. 100 mL medium requires 22.22 mg, which is 4.44 mL of solution (22.22/30×6 mL=4,44 mL).

5.0 Peptide Stimulation Assay

5.1 Assay is performed on Day 14 after initiation of cell differentiation. On day prior to stimulation assay, replace growth medium+insulin with medium prepared in 4.1 (a-MEM+0.5% BSA) and return plates to incubator.

5.2 On Day of assay prepare peptide solutions as in 4.2 and IBMX as in 4.3.

5.3 Replace medium with the same medium supplemented with 1 mM IBMX (as in 4.3) and allow cells to equilibrate in incubator for 10 minutes.

5.4 Add increasing concentrations of peptides (or none), 33 ul per well, swirl gently to mix, and place plates in incubator for 4 hours.

5.5 At the end of the incubation remove media from wells and store triplicate aliquots in −20° C. freezer for measurement of leptin and triglycerides.

5.6 Stain adipocytes in wells with Oil Red O as in “Oil Red O Staining of Adipocyte” document

5.7 After eluting the stain, remove cells from wells as in 6.0 for measuring total protein.

Table 5: Effect of alpha-MSH or desacetyl-alpha-MSH on leptin production in differentiated 3T3L1 adipocytes.

Leptin results are from 2 separate 4-hour peptide stimulation assays of triplicate incubation wells for each dose. For each assay, leptin was measured in duplicate samples from triplicate incubation wells and data was normalised to percentage of control (results with no added peptide). Mean control leptin results for the 2 assays were 930±47 pg/mL and 535±61 pg/mL. Data in the table is the combined normalised results from the 2 assays, showing mean % of control±SEM.

| | | | | | Leptin Response with | | |
|---|---|---|---|---|---|---|---|
| PEPTIDE | | Leptin Response with aMSH | | | desacetyl aMSH | | |
| DOSE | | Mean | SEM | n | Mean | SEM | n |
| 0 | (Control) | 100.0 | | 6 | 100.0 | | 6 |
| 1 | nM | 100.8 | 4.5 | 11 | 95.4 | 2.3 | 11 |
| 10 | nM | 107.4 | 6.4 | 10 | 96.3 | 3.6 | 12 |
| 100 | nM | 100.9 | 5.7 | 9 | 97.7 | 5.0 | 12 |
| 1000 | nM | 98.0 | 3.3 | 12 | 109.4 | 6.5 | 12 |

There is a trend for desacetyl-alpha-MSH but not alpha-MSH to reduce leptin production over this time period. The triglyceride levels did not appear to change (see Table 6) and therefore this reduction in leptin production may reflect a reduction in leptin gene transcription.
Leptin Assays: Quantikine M kit (R & D Systems Inc. UK # MOB00 Abingdon, Oxon) and DSL kit (DSL #10-24100, Australia PTY Ltd, NSW, Australia) were used. Both are specific for murine leptin, validated for use with cell culture medium, and showed a similar result for an in-house quality control pooled murine plasma sample. The Quantikine M kit is preferred as it is more sensitive and precise.
Table 6: Effect of alpha-MSH or desacetyl-alpha-MSH on triglyceride release from 3T3 L1 adipocytes in the 2 peptide stimulation assays in Table 5.

Results in each peptide stimulation assay were normalised to percentage of control. Data in the table is the combined normalised results from the 2 assays, showing mean % of control±SEM.

| | | |
|---|---|---|
| | | TRIGLYCERIDE |
| | | RESPONSE (% |
| PEPTIDE | PEPTIDE | OF CONTROL) |

| ADDED | DOSE | | MEAN | SEM |
|---|---|---|---|---|
| NONE (CONTROL) | 0 | | 100 | |
| AMSH | 100 | nM | 110.5 | 5.6 |
| | 1000 | nM | 105.7 | 9.3 |
| DA MSH | 100 | nM | 111.4 | 6.7 |
| | 1000 | nM | 110.0 | 7.2 |

Table 7: Effect of Different Ratios of Alpha-MSH and Desacetyl-Alpha-MSH on Leptin Production in Differentiated 3T3L1 Cells.

In one of the two peptide simulation assays described in Table 5, 7 different peptide ratios (as indicated in the table below) were added to triplicate wells. Leptin was measured in duplicate samples from each well. Data shown is mean leptin level±SEM (pg/mL) from the single dose concentrations of each peptide and the 3 ratios.

| | | | | |
|---|---|---|---|---|
| Peptide | Concentration | Mean | SEM | n |

| | | | | | |
|---|---|---|---|---|---|
| aMSH | 1 | nM | 544.2 | 54.6 | 5 |
| | 100 | nM | 547.0 | 52.9 | 6 |
| desacetyl | 1 | nM | 480.4 | 14.5 | 5 |
| aMSH | 100 | nM | 528.0 | 53.9 | 6 |
| (da MSH) | | | | | |

| | | | |
|---|---|---|---|
| 1 nM aMSH + 100 nM da MSH | 417.8 | 68.0 | 5 |
| 100 nM aMSH = 1 nM da MSH | 562.3 | 68.5 | 6 |
| 1 nM aMSH + 1 nM da MSH | 575.0 | 29.2 | 6 |

Compared to 1 nM alpha-MSH and 100 nM desacetyl-alpha-MSH, the ratio of 100 nM desacetyl-alpha-MSH/1 nM alpha-MSH appears to reduce leptin production. Therefore an abundance of desacetyl-alpha-MSH may lead to reduced leptin gene transcription.

While 1 nM desacetyl-alpha-MSH appears to reduce leptin production, the ratio of 1 nM desacetyl-alpha-MSH/100 nM alpha-MSH does not appear to reduce leptin production and neither does 100 nM alpha-MSH alone. Therefore an abundance of alpha-MSH may prevent desacetyl-alpha-MSH from reducing leptin gone transcription.

While 1 nM desacetyl-alpha-MSH appears to reduce leptin production, the ratio of 1 nM desacetyl-alpha-MSH/1 nM alpha-MSH does not appear to reduce leptin production and neither does 1 nM alpha-MSH alone. Therefore an equimolar concentration of alpha-MSH may be sufficient to prevent desacetyl-alpha-MSH from reducing leptin gene transcription.

It will be understood from the foregoing that either a reduction in the level of alpha-MSH or the increase in the level of desacetyl-alpha-MSH will result in a higher desacetyl-alpha-MSH : alpha-MSH ratio. Further, a reduction in the level of alpha-MSH or desacetyl-alpha-MSH individually, with respect to sex and age matched reference ranges, may also be used effectively in the methods of the present invention. Not wishing to be bound by any particular theory, it is likely that desacetyl-alpha-MSH alone, at levels above a particular threshold, would be useful in the methods of the present invention.

Although the invention has been described with reference to specific examples, it will be appreciated by those skilled in the art that the invention may be embodied in many other forms.

1. Method for assessing feeding and/or weight gain pattern in a subject comprising the measurement of a melanocortin peptide in a sample obtained from said subject and comparison of the measured value with a reference value.

2. Method for predicting risk of obesity in a subject comprising the measurement of a melanocortin peptide in a sample obtained from said subject and comparison of the measured value with a reference value

3. Method for diagnosing imbalance in energy homeostasis in a subject comprising the measurement of a melanocortin peptide in a sample obtained from said subject and comparison of the measured value with a reference value.

4. Method for diagnosing obesity in a subject comprising the measurement of a melanocortin peptide in a sample obtained from said subject and comparison of the measured value with a reference value

5. Method for screening medicaments for the adverse reactions of imbalance in energy homeostasis, feeding/weight gain patterns or obesity in a subject to whom the medicament has been administered comprising the measurement of a melanocortin peptide in a sample obtained from said subject, and comparison of the measured value with a reference value.

6. Method for screening foods and/or diets for the adverse reactions of imbalance in energy homeostasis, feeding/weight gain patterns or obesity in a subject to whom the medicament has been administered comprising the measurement of a melanocortin peptide in a sample obtained from said subject, and comparison of the measured value with a reference value.

7. A method according to any one of claims 1 to 6, wherein the melanocortin peptide measured is α-MSH or desacetyl-α-MSH.

8. Method for assessing feeding and/or weight gain pattern in a subject comprising the measurement of at least two melanocortin peptides in a sample obtained from said subject, the calculation of the ratio of the measured melanocortin peptides and comparison of the value of the ratio with a reference value.

9. Method for predicting risk of obesity in a subject comprising the measurement of at least two melanocortin peptides in a sample obtained from said subject, the calculation of the ratio of the measured melanocortin peptides and comparison of the value of the ratio with a reference value.

10. Method for diagnosing obesity in a subject comprising the measurement of at least two melanocortin peptides in a sample obtained from said subject, the calculation of the ratio of the measured melanocortin peptides and comparison of the value of the ratio with a reference value.

11. Method for diagnosing imbalance in energy homeostasis in a subject comprising the measurement of at least two melanocortin peptides in a sample obtained from said subject, the calculation of the ratio of the measured melanocortin peptides and comparison of the value of the ratio with a reference value.

12. Method for screening medicaments for the adverse reactions of imbalance in energy homeostasis, feeding/weight gain patterns or obesity in a subject to whom the medicament has been administered comprising the measurement of at least 2 melanocortin peptides in a sample obtained from said subject, the calculation of the ratio of the measured melanocortin peptides, and comparison of the value of the ratio with a reference value.

13. Method for screening foods and/or diets for the adverse reactions of imbalance in energy homeostasis, feeding/weight gain patterns or obesity in a subject to whom the medicament has been administered comprising the measurement of at least 2 melanocortin peptides in a sample obtained from said subject, the calculation of the ratio of the measured melanocortin peptides, and comparison of the value of the ratio with a reference value.

14. A method according to any one of claims 8 to 13, wherein the melanocortin peptide ratio calculated is the ratio of desacetyl-α-MSH to α-MSH.

15. A method according to any one of claims 1 to 14, wherein the melanocortin peptides are measured by a biological response system and wherein the resulting profile of response parameters is predictive of the risk of developing obesity or diagnostic of obesity, imbalance in energy homeostasis or disturbance in feeding/weight gain patterns.

16. Method of assessing risk of developing obesity, diagnosing obesity or diagnosing an imbalance in energy homeostasis or disturbance in feeding/weight gain patterns in a subject, comprising:
a. measuring the amount of α-MSH and desacetyl-α-MSH in a sample obtained from the subject, either directly or by subtraction of one of the amount of α-MSH or desacetyl-α-MSH from a measured amount of total MSH in the sample,
b. calculating the ratio of the amounts of desacetyl-α-MSH to α-MSH.
c. comparing the ratio of desacetyl-α-MSH to α-MSH with a reference ratio.

17. A method according to any one of claims 1 to 16, wherein the measurement is quantitative.

18. A method according to any one of claims 1 to 17, wherein α-MSH and desacetyl-α-MSH are separated from the sample before measurement.

19. A method according to claim 18, wherein α-MSH and desacetyl-α-MSH are separated by a procedure selected from the group consisting of chromatography, electrophoresis, immunocapture and affinity capture.

20. A method according to any one of claims 1 to 14 or 16 to 19, wherein the melanocortin peptide is measured by an immuno-assay.

21. Method of monitoring treatment for obesity or for imbalance in energy homeostasis and/or disturbance in feeding/weight gain pattern in a subject comprising contacting a sample obtained from the subject having such treatment with a biological response system wherein the resulting profile of response parameters is indicative of the effect of such treatment on obesity or imbalance in energy homeostasis and/or disturbance in feeding/weight gain pattern.

22. Method of assessing the risk of developing obesity or developing and/or having an imbalance in energy homeostasis and/or disturbance in feeding/weight gain pattern in a subject comprising analysing the profile of response parameters in a sample from a test subject by comparing it with
(i) the profile of a sample from a normal subject and
(ii) the profile of a sample from an obese subject or a subject with an imbalance in energy homeostasis and/or disturbance in feeding/weight gain pattern,
wherein resemblance of the profile of the sample obtained from the test subject to that of the profile in (ii) above, is indicative of that subject being at risk of developing obesity or developing and/or having an imbalance in energy homeostasis and/or disturbance in feeding/weight gain pattern.

23. A method according to any one of claims 1 to 22, wherein the subject is a mammal.

24. Method of determining the melanocortin peptide status of a sample comprising contacting the sample with a biological response system wherein the resulting profile of response parameters produced by the biological response system indicates the melanocortin peptide status of the sample.

25. A method according to any one of claims 1 to 24, wherein the sample is a biological fluid selected from the group consisting of whole blood, plasma, serum, saliva, sweat, urine, amniotic fluid, cord blood and cerebrospinal fluid.

26. Method of screening for a compound which acts as agonist or antagonist of a melanocortin receptor comprising treating a biological response system with a test compound and measuring the resulting profile of response parameters that are indicative of agonist or antagonist activity to the melanocortin receptor.

27. Method of screening for a compound that is useful in the treatment of obesity comprising exposing a biological response system to a test compound and measuring the resulting profile of response parameters that are indicative of the desired response for the treatment of obesity.

28. Method of screening for a compound that is useful in the treatment of an imbalance in energy homeostasis or a disturbance in feeding/weight gain patterns comprising exposing a biological response system to a test compound and measuring the resulting profile of response parameters that are indicative of the desired response for the treatment of an imbalance in energy homeostasis or a disturbance in feeding/weight gain patterns.

29. A method according to any one of claims 15 or 21 to 28, wherein the biological response system is an in vitro cell, organ or tissue sample, or whole animal capable of responding to melanocortin peptides.

30. A method according to claim 29, wherein the in vitro cell is selected from the group consisting of primary osteoblasts, osteosarcoma cell line, hypothalamic cell line, adipocytes, myocytes, melanoma cells and anterior pituitary cells.

31. A method according to claim 29, wherein the organ or tissue sample is that of hypothalamus.

32. A method according to any one of claims 15 or 21 to 31, wherein the profile of response parameters measured comprise one or more proteins or cellular events which differentiate between normal subjects and those at risk of developing obesity or having obesity, or those with an imbalance in energy homeostasis, or disturbance in feeding/weight gain patterns.

33. A method according to claim 32, wherein the one or more proteins are selected from the group consisting of heat shock protein homologue, glyceraldehyde-3-phosphate-dehydrogenase, aldo-keto reductase, citrate synthase, creatine kinase, pyruvate synthase alpha-chain, f1 ATPase beta-chain, tubulin beta-chain, proteins involved in the melanocortin peptidergic axis, proteins involved in signalling pathways and membrane-bound proteins.
